(19) United States
(12) Patent Application Publication
(10) Pub. No.: US 2019/0322983 A1
(43) Pub. Date: Oct. 24, 2019

(54) ENGINEERED TREG CELLS

(71) Applicant: Memorial Sloan Kettering Cancer Center, New York, NY (US)

(72) Inventors: Alexander Y. Rudensky, New York, NY (US); Takatoshi Chinen, New York, NY (US)

(21) Appl. No.: 16/310,668

(22) PCT Filed: Jun. 15, 2017
(86) PCT No.: PCT/US17/37794

§ 371 Date: Dec. 17, 2018

Related U.S. Application Data
(60) Provisional application No. 62/351,104, filed on Jun. 16, 2016.

(51) Int. Cl. C12N 5/0783; C07K 14/47; A61K 35/17; A61K 48/00

(52) U.S. Cl. CPC C12N 5/0637; C07K 14/4702; A61K 38/00; A61K 48/005; C12N 2510/00; A61K 35/17

(57) ABSTRACT

The present invention provides, among other things, methods and compositions for modulating or treating inflammatory and autoimmune diseases, disorders, and conditions. The present invention is based, in part, on the surprising discovery that engineered regulatory T-cells characterized by constitutive STAT activity are efficacious in treating disease.

### GOVERNMENT SUPPORT

This invention was made with government support under CA008748, AI034206 and GM07739 awarded by the National Institutes of Health. The government has certain rights in the invention.

### BACKGROUND

With advancements in understanding of immune systems additional avenues for therapeutics arise. There is a need to identify novel compositions and methods of treatment to treat disease using the immune system.

### SUMMARY

The present disclosure encompasses the recognition that novel therapies can be developed to treat diseases, disorders, or conditions through the engineering of cells of the immune system. In some embodiments, the present disclosure recognizes that some diseases, disorders, or conditions, e.g. inflammatory and autoimmune diseases, can be a result of an overactive and or self-reactive immune system. In some embodiments, the present disclosure recognizes regulatory T-cells (Treg) can be a useful tool to regulate an overactive and or self-reactive immune system. In some embodiments, the present disclosure relates to engineering Treg cells to treat diseases, disorders, or conditions, e.g. inflammatory and autoimmune diseases. In some embodiments, the present disclosure recognizes that engineering a Treg cell to be independent of a need for IL-2 signaling for stimulation can provide a novel therapeutic for the treatment of inflammatory and autoimmune diseases.

In some embodiments, the present disclosure relates to an engineered regulatory T cell characterized by constitutive STAT activity. In some embodiments, the present disclosure provides an engineered Treg cell that expresses a constitutively active STAT protein. In some embodiments, a constitutively active STAT protein is a phosphorylated protein (e.g., a constitutively phosphorylated protein). In some embodiments, a Treg cell as described herein is engineered to constitutively express a STAT protein. In some embodiments, a Treg cell as described herein is engineered to constitutively activate a STAT protein (e.g., by constitutively converting a STAT protein from an inactive to an active form, for example, by phosphorylation). In some embodiments, an engineered Treg cell characterized by constitutive STAT activity contains a higher and/or more temporally consistent level and/or activity of a particular STAT protein, or active form thereof, as compared with an appropriate reference Treg cell (e.g., an otherwise comparable Treg cell lacking the relevant engineering) under comparable conditions.

In some embodiments, an engineered Treg cell characterized by constitutive STAT activity as described herein also expresses a chimeric antigen receptor. Alternatively or additionally, in some embodiments, an engineered Treg cell characterized by constitutive STAT activity as described herein also expresses an endogenous T-cell receptor.

In some embodiments, the present disclosure provides technologies for treating one or more diseases, disorders, or conditions. In some particular embodiments, the present disclosure relates to treatment of inflammatory or autoimmune diseases.

In some embodiments, the present disclosure provides methods that include a step of engineering one or more Treg cells obtained from a patient sample to achieve constitutive STAT activity in the engineered Treg cell (e.g., as compared with an otherwise comparable Treg cell lacking the engineering). In some embodiments, a method of treatment as described herein may be or comprise administration of an engineered Treg cell as described herein (i.e., an engineered Treg cell characterized by constitutive STAT activity).

### BRIEF DESCRIPTION OF THE DRAWING

FIG. 1, comprising panels (a) through (j) demonstrates IL-2Rβ is indispensable for Treg cell function. Panel (a) shows the histopathology of indicated organs of 5-wk-old Foxp3^{Cre}Il2rb^{fl/wt} and Foxp3^{Cre}Il2rb^{fl/fl} mice. Scale bar, 100 Representative images of 5 vs. 5 mice analyzed are shown. Panel (b) shows lymph node (LN) cellularity of 5-wk-old Foxp3^{Cre}Il2rb^{fl/wt} and Foxp3^{Cre}Il2rb^{fl/fl} mice. Panel (c) shows flow cytometric analysis of cytokine production by splenic CD4+ Foxp3− cells of 5-wk-old Foxp3^{Cre}Il2rb^{fl/wt} and Foxp3^{Cre}Il2rb^{fl/fl} mice stimulated for 5 hr with anti-CD3/CD28. Panel (d) shows flow cytometric analysis of cell-surface expression of indicated IL-2R subunits by CD4+ Foxp3+ cells from Foxp3^{Cre}Il2rb^{fl/wt} (blue) and Foxp3^{Cre}Il2rb^{fl/fl} (red) mice. Representative images of 5 vs. 5 mice analyzed are shown. Panel (e) shows flow cytometric analysis of STAT5 phosphorylation in IL-2Rβ-deficient Treg cells. Splenocytes from Foxp3^{Cre}Il2rb^{fl/wt} (blue) and Foxp3^{Cre}Il2rb^{fl/fl} (red) mice were cultured with or without recombinant murine IL-2 (rmIL-2; 1,000 U/ml) for 20 min, and intracellular levels of tyrosine phosphorylated STAT5 in CD4+YFP+(Foxp3+) cells were analyzed by flow cytometry. Representative images of 5 vs. 5 mice analyzed are shown. Panel (f) shows flow cytometric analyses of the frequencies of Treg cells among CD3+CD4+ cells (left graph) and Foxp3 expression levels (MFI: mean fluorescence intensity) (right graph) in the LNs of 5-wk-old Foxp3^{Cre}Il2rb^{fl/wt} and Foxp3^{Cre}Il2rb^{fl/fl} mice. Panel (g) shows representative flow cytometric analyses of Treg cells in healthy heterozygous female Foxp3^{Cre/wt}Il2rb^{fl/wt} and Foxp3^{Cre/wt}Il2rb^{fl/fl} mice. Cells isolated from the indicated organs were analyzed for Foxp3 and YFP expression. YFP (Cre) expression and intracellular Foxp3 staining identified Treg cells with or without YFP-Cre expression. Gates shown are for CD3+CD4+ cells. Panel (h) shows the frequencies of Foxp3+ cells among CD3+CD4+ cells (upper panel) and the frequencies of Cre expressing cells among Foxp3+ cells (lower panel) in the indicated organs of 3-wk-old heterozygote female Foxp3^{Cre/wt}Il2rb^{fl/wt} (black) and Foxp3^{Cre/wt}Il2rb^{fl/fl} (red) mice. Panel (i) shows Foxp3 expression levels (MFI) in YFP-Foxp3+(upper panel) and YFP+ Foxp3+(lower panel) cells in the indicated organs of 3-wk-old Foxp3^{Cre/wt}Il2rb^{fl/wt} (black) and Foxp3^{Cre/w}tIl2rb^{fl/fl} (red) mice. Panel (j) shows expression levels of indicated markers (MFI) and the frequencies of CD103+ cells in YFP+ Foxp3+ cells in the indicated organs of 3-wk-old Foxp3^{Cre/w}tIl2rb^{fl/wt} (black) and Foxp3^{Cre/wt}Il2rb^{fl/fl} (red) mice.

FIG. 2, comprising panels, (a) through (k), demonstrates restoration of the suppressor activity of IL-2R-deficient Treg cells in the presence of a constitutively active form of STAT5. Panel shows (a) a schematic of the targeting construct. Panel (b) shows rescue of wasting disease in Foxp3^{Cre}Il2rb^{fl/fl} mice upon expression of a conditional ROSA26^{Stat5bCA} transgene. Mice were analyzed at 4 wk of age. Representative picture of more than 10 Foxp3^{Cre}Il2rb^{fl/fl} vs. 10 Foxp3^{Cre}Il2rb^{fl/fl} ROSA26^{Stat5bCA} mice analyzed are shown. Panel (c) shows frequency of Foxp3+ cells among CD3+CD4+ cells and the levels of CD122 and CD25 expression on CD3+CD4+ Foxp3+ cells. Data are representative of two independent experiments. Panel (d) shows flow cytometric analysis of STAT5 phosphorylation in Treg cells. LN cells isolated from the indicated mice were unstimulated (unstim) or stimulated with rmIL-2 (1,000 U/ml) for 20 min, and intracellular levels of tyrosine phosphorylated STAT5 in CD4+YFP+(Foxp3+) cells were analyzed. Data are representative of two independent experiments. Panel (e) shows rescue of wasting disease in Foxp3^{Cre}Il2ra^{fl/fl} mice in the presence of ROSA26^{Stat5bCA} transgene. Mice were analyzed at 4 wk of age. Representative picture of more than 10 Foxp3^{Cre}Il2ra^{fl/fl} vs. 10 Foxp3^{Cre}Il2ra^{fl/fl} ROSA26^{Stat5bCA} mice analyzed are shown. Panel (f) shows in vitro IL-2 capture assay. GFP(YFP)+ Treg cells and GFP(YFP)− non-Treg cells from the indicated mice were sorted and cultured for 2 hrs with recombinant human IL-2 (hIL-2). The amount of residual hIL-2 in the media after 2 hrs were measured using flow cytometry-based bead array analysis and shown as percent value. Representative data of two independent experiments are shown. Panel (g) shows cell numbers of CD3+CD4+ Foxp3− CD44^{hi}, CD44hi, CD3+CD8+CD62L^{lo}CD44^{hi}, and CD3+CD8+CD62L^{hi}CD44^{hi} cells in the LNs of 2 wk old mice as determined by flow cytometry. Foxp3^{Cre}Il2rb^{wt/wt} (black), Foxp3^{Cre}Il2rb^{fl/f}l (red), and Foxp3^{Cre}Il2rb^{fl/fl}ROSA26^{Stat5bCA} (blue). Data are representative of two independent experiments. Panel (h) T shows frequency of naïve (CD62LhiCD44lo) cells among CD3+CD4+ Foxp3− and CD3+CD8+ Foxp3− cells (left two panels) and the cell numbers of CD44hi activated CD3+CD4+ Foxp3− and CD3+CD8+ Foxp3− cells (right two panels) in the LNs of indicated mice as determined by flow cytometry. The mice were either treated with anti-IL-2 neutralizing antibodies or control IgG for 2 wks starting from 7 days after birth. Representative data of two independent experiments are shown. Panel (i) shows analysis of the ability of IL-2R-sufficient and -deficient Treg cells to suppress the expansion of naïve and activated/memory CD4+ and CD8+ T cells. CD4+ Foxp3-CD62LhiCD44lo (CD4 naïve), CD8+ Foxp3-CD62LhiCD44lo (CD8 naïve), and CD8+ Foxp3-CD62LhiCD44hi (CD8 memory) T cells were sorted from wild type (Foxp3Cre) mice and adoptively transferred (1×106 cells each) into T cell-deficient (Tcrb−/− Tcrd−/−) mice together with Treg cells (2×105 cells) separately sorted from the indicated mice. CD4+ Foxp3− and CD8+ Foxp3− T cell numbers in the recipients 3 wks after transfer are shown. Panel (j) shows analysis of susceptibility of CD4+ and CD8+ T cells expressing a constitutively active form of STAT5 to Treg mediated suppression. CD4+ Foxp3− and CD8+ Foxp3− T cells were sorted from Foxp3^{Cre}ROSA26S^{tat5bCA} mice and treated in vitro with TAT-Cre recombinase to induce STAT5bCA expression in non-Treg CD4+ and CD8+ T cells. Recombination efficiency was approximately 30% for both cell subsets. The treated CD4+ Foxp3− and CD8+ Foxp3− T cells (1×10⁶ cells each) were transferred together into T cell-deficient (Tcrb−/−Tcrd−/−) recipients without Treg cells (red bars) or with 2×10⁵ control (black bars) or STAT5bCA-expressing Treg cells (blue bars) sorted from Foxp3^{Cre} or Foxp3^{Cre}ROSA26^{Stat5bCA} mice, respectively. The recipients were analyzed 3 wks after transfer. The frequencies of STAT5bCA-expressing CD4+ and CD8+ Teff cells within total CD4+ and CD8+ effector T cell subsets are shown. Panel (k) shows the numbers of IFNγ-producing CD4+ and CD8+ T cells in the recipient mice described in (j). As a control, CD4+ Foxp3− and CD8+ Foxp3 T cells sorted from Foxp3^{CreROSA26WT} mice (WT) mice were similarly treated with membrane-permeable TAT-Cre protein and transferred with or without Treg cells to assess the susceptibility of STAT5bCA-non-expressing effector T cells to Treg mediated suppression (open bars). The lower two graphs are shown in % calculated from the same data sets. Data are representative of two independent experiments. Each dot represents a single mouse. Error bars indicate mean+/−S.E.M (c, d, g, h, i, j, k).

FIG. 3, comprising panels (a) through (g), demonstrates increased proliferative and suppressor activity of Treg cells expressing a constitutively active form of STAT5. Panel (a) shows frequency of Foxp3+ cells among CD3+CD4+ cells (upper graph) and expression levels of Foxp3 in CD3+CD4+ Foxp3+ cells (lower graph) in the indicated organs were determined by flow cytometry. Sp: spleen, SILPL: small intestine lamina propria lymphocytes. Representative data of two independent experiments are shown. Panel (b) shows representative flow cytometric analysis of splenocytes showing the increase of CD25hiFoxp3hi population in CD4+ T cells of Foxp3^{Cre-ERT2}ROSA26^{Stat5bCA} mice. Panel (c) shows representative flow cytometric analysis of splenic Treg cells in Foxp3^{Cre-ERT2} and Foxp3^{Cre-ERT2}ROSA26^{Stat5bCA} mice. Cells were stained for CD62L, CD44, KLRG-1, ICOS, CTLA-4, and GITR. Panel (d) shows flow cytometric analyses of splenic Treg cells for the expression levels of the indicated markers in the indicated mice. Representative data of two independent experiments are shown. Panel (e) shows representative flow cytometric analysis of splenic CD3+CD4+ Foxp3− (upper panels) and CD3+CD8+ Foxp3− (lower panels) cells in Foxp3^{Cre-ERT2} and Foxp3^{Cre-ERT2}ROSA26^{Stat5bCA} mice. Panel (f) shows flow cytometric analysis of expression of CD80 and CD86 on DCs (CD11c+MHC class IIhi) and B cells (B220+CD11c−) in the LNs of the indicated mice. Representative data of two independent experiments are shown. Panel (g) shows serum and fecal IgA levels in the indicated mice as determined by ELISA. Foxp3^{Cre-ERT2} (black dots) and ^{Fov3Cre-ERT2}ROSA26^{Stat5bCA} (blue dots) mice were analyzed three months after a single tamoxifen treatment. Each dot represents a single mouse. Error bars indicate mean+/−S.E.M (a, d, f, g).

FIG. 4, comprising panels a through e, demonstrates potent suppressor function of Treg cells expressing a constitutively active form of STAT5. Panel (a) shows analysis of EAE in the presence of STAT5bCA expressing and control Treg cells in Foxp3^{Cre-ERT2} (black) and Foxp3^{Cre-ERT2}ROSA26^{Stat5bCA} (blue) mice. EAE was induced upon immunization with MOG peptide in CFA. Average disease scores of the indicated mice (n=10 for each group). Error bars indicate +/−S.E.M. Representative data of two independent experiments are shown. Panel (b) shows frequency of Foxp3+ cells among brain-infiltrating CD3+CD4+(left graph) and CD3+CD8+(right graph) cells in mice shown in (a) as determined by flow cytometry. Panel (c) shows the numbers of the indicated brain-infiltrating cell subsets in mice shown in (a) as determined by flow cytometry. Panel (d) shows analysis of T cell responses against Listeria monocytogenes in the indicated mice. Spleen T cell responses were analyzed on day 8 after Listeria infection. The frequencies of Foxp3+ Treg cells among CD3+CD4+ cells (left). The frequencies of IFNγ (middle) and TNFα (right graph) producing CD4+TCRβ+ Foxp3− cells were analyzed after 5 hr in vitro re-stimulation with heat-killed Listeria in the presence of DCs. Pooled data from four independent experiments are shown. Panel (e) shows analysis of anti-viral T cell responses in the indicated mice infected with non-replicating vaccinia virus. Spleen T cell responses were analyzed on day 8 after infection. Vaccinia B8R peptide-specific CD8+ T cells were detected by flow cytometry using H-2Kb-B8R tetramer staining (left graph). IFNγ production by CD8+ Foxp3− (middle) and CD4+ Foxp3− (right graph) cells was determined by flow cytometry after a 5 hr in vitro stimulation with B8R peptide or a mixture of three vaccinia virus-specific peptides (ISK, A33R, and B5R). Representative data of two independent experiments are shown. Foxp3Cre-ERT2 (black) and Foxp3Cre-ERT2ROSA26Stat5bCA (blue) mice two to three months after a single tamoxifen treatment were challenged with the indicated inflammatory agents. Each dot represents an individual mouse (b, c, d, e). Error bars indicate mean+/−S.E.M.

FIG. 5, comprising panels (a) through (f), demonstrates RNA-seq analysis of Treg cells expressing a constitutively active form of STAT5. Panel (a) shows principal component analysis of RNA-seq datasets, using the top 15% of genes with the highest variance. Each dot corresponds to an RNA sample from a single mouse. Panel (b) shows plots of gene expression (as log 2 normalized read count) in control Treg vs. STAT5bCA expressing Treg cells. The diagonal lines indicate fold change of at least 1.5× or 0.67× fold. Significantly up- and down-regulated genes (defined as genes with at least 1.5× or 0.67× fold change, adjusted P-value≤0.05, and expression above a minimal threshold based on the distribution of all genes) are colored red or blue, respectively, and their numbers are shown. Panel (c) shows a heat map of selected genes. For each condition, 3 replicates are shown in order. The values indicate FDR-adjusted P-values between control Treg and STAT5bCA expressing Treg cells. Panel (d) shows empirical cumulative distribution function (ECDF) for the log 2 fold change of all expressed genes in STAT5bCA versus control Treg, is plotted along with ECDFs for the subsets of genes up- or down-regulated by inflammatory activation in Treg cells³³ (upper graph), or the subsets of genes up- or down-regulated in a TCR-dependent manner in CD44hi Treg cells³⁴ (lower graph). FDR-adjusted P-values were computed using the two-sided Kolmogorov-Smirnov test. Panel (e) shows Signaling Pathway Impact Analysis (SPIA) of KEGG pathways. The 6 most statistically significant pathways that show enrichment among differentially expressed (DE) genes in STAT5bCA versus control Treg cells are shown. The net pathway perturbation indicates the status of the pathway (positive=activated; negative=inhibited) based on the activating or inhibitory relationships of DE genes within the pathway. The size of the red circle is proportional to the degree of enrichment, and the FDR-adjusted global P-value reflecting both enrichment and perturbation is shown. Panel (f) shows network analysis of GO term enrichment among significantly upregulated genes in STAT5bCA Treg versus control Treg cells. Upregulated genes were analyzed for over-represented GO terms using BiNGO in Cytoscape, and the resulting network was calculated and visualized using EnrichmentMap. Groups of similar GO terms were manually circled. Edge thickness and color are proportional to the similarity coefficient between connected nodes. Node color is proportional to the FDR-adjusted P-value of the enrichment. Node size is proportional to gene set size. For RNA-seq analyses splenic CD4+ Foxp3+ Treg and CD4+ Foxp3-CD62LhiCD44lo Tnaïve cells were FACS purified from Foxp3^{Cre-ERT2}ROSA26^{Stat5bCA} (STAT5bCA) and Foxp3^{Cre-ERT2} (control) mice 4 months after tamoxifen treatment.

FIG. 6, comprising panels (a), (b), and (c), demonstrates augmented STAT5 signaling in Treg cells increases the conjugate formation between Treg cells and DCs and potentiates suppressor function in a TCR independent manner. Panel (a) shows analysis of in vitro conjugate formation between T cells and DCs. For conjugate formation assessment, FACS-sorted, CFSE-labeled T cells (Treg and non-Treg cells) from the indicated mice were co-cultured with graded numbers of MACS-sorted, CellTrace Violet-labeled CD11c+ DCs from C57BL/6J mice for 150 to 720 min in the presence or absence of rmIL-2 (100 IU/ml). Each dot represents a flow cytometric analysis of conjugate formation in a single well. The statistical data analysis was performed by modified analysis of covariance (ANCOVA) using Prism software package. **, P<0.01; ***, P<0.001; NS, not significant. Representative data of threeindependent experiments are shown. Panel (b) shows expression of a constitutively active form of STAT5 potentiates Treg cell suppressor function in the absence of TCR signaling. Foxp3^{Cre-ERT2} (solid circle), Foxp3^{Cre-ERT2}ROSA26^{Stat5bCA} (bordered circle), Foxp3^{Cre-ERT2}Tcra^{fl/fl} (solid triangle), and ^{Foxp3Cre-ERT2}Tcraf^{l/fl}ROSA26^{Stat5bCA} mice (bordered triangle) were treated with tamoxifen for 2 wks and T cell activation, proliferative activity and pro-inflammatory cytokine production were assessed by flow cytometry. LN cellularity (left), and the frequencies of CD44hi (middle left), Ki-67+ cell (middle right), IFNγ+ producing cells (right) among CD4+ Foxp3− cells are shown. Each dot in graphs represents a single mouse. Error bars indicate mean+/−S.E.M. Representative data of three independent experiments are shown. Panel (c) shows the frequencies of Treg cells and ecpssion of certain molecules. WT CD4+ Foxp3− and CD8+ Foxp3− T cells (5×10⁵ cells each) were transferred into Tcrb^{-/-}Tcrd^{-/-} recipients together with Treg cells (3×10⁵ cells) sorted from the indicated mice that had been treated with tamoxifen for 2 wks. TCR-ablated Treg cells were FACS purified based on the expression of TCR. TCR-sufficient Treg cells were sorted from the control (Foxp3^{Cre-ERT2)} mice. The recipients were analyzed 3 wks after transfer. The frequencies of Treg cells in the recipients and the expressions of indicated molecules in Treg cells are shown in the first five panels (left to right). The right two panels show the numbers of CD4+ Foxp3- and CD8+ Foxp3− T cells. Representative data of two independent experiments are shown.

FIG. 7, comprising panels (a) through (c), demonstrates IL-2 maintains both CD62LhiCD44lo and CD62LloCD44hi Treg cell subsets. Panel (a) shows flow cytometric analyses of mice shown in FIG. 1j were performed by gating on CD62LhiCD44lo (upper panels) and CD62LloCD44hi (lower panels) YFP+ Foxp3+ Treg cell subsets. Representative data of two independent experiments are shown. Panel (b) shows representative flow cytometric analyses of the expressions of CD62L and CD44 in CD3+CD4+ Foxp3+(upper panels) and frequencies of Foxp3+ cells among CD3+CD4+ cells (lower panels) in the spleen and small intestine lamina propria lymphocytes (SILPL) of 5-wk-old Foxp3^{Cre}Il2rb^{fl/wt} and Foxp3^{Cre}Il2rb^{fl/fl} mice. The right graph shows the summary data of flow cytometry plots. Panel (c) shows flow cytometric analyses of the indicated markers for splenic CD3+CD4+ Foxp3+ cells of 5-wk-old Foxp3^{Cre}Il2rb^{fl/wt} and Foxp3^{Cre}Il2rb^{fl/fl} mice. Representative data of three independent experiments are shown. Each dot in graphs represents a single mouse. Error bars indicate mean+/−S.E.M (a, b, c).

FIG. 8, comprising panels (a) through (h), demonstrates IL-2Rα and STAT5 are indispensable for Treg cell function. Panel (a) shows lifespan of Foxp3^{Cre}Il2ra^{fl/fl} (solid; n=25) and control Foxp3^{Cre}Il2ra^{fl/wt} (dotted; n=20) mice. Panel (b) shows analysis of LN cellularity, Foxp3 expression levels (MFI) and frequencies of Foxp3+ Treg cells among CD3+CD4+ cells (upper graphs) and pro-inflammatory cytokine production by CD4+ Foxp3− and CD8+ Foxp3− cells (lower graphs) in 4-wk-old Foxp3^{Cre}Il2ra^{wt/wt} and Foxp3^{Cre}Il2ra^{fl/fl} mice. Each dot represents a single mouse. Error bars indicate mean+/−S.E.M. Representative data of two independent experiments are shown. Panel (c) shows histopathology analysis of Foxp3^{Cre}Il2ra^{fl/fl} mice. H&E staining of the formalin-fixed tissue sections of the indicated organs of 4-wk-old mice. Scale bar, 100 Representative images of 3 mice analyzed are shown. Panel (d) shows epresentative flow cytometric analysis of Foxp3 and CD25 expression in CD4 T cell subset in the LNs of 6-wk-old Foxp3^{Cre}Stat5a/b^{wt/wt} and Foxp3^{Cre}Stat5a/b^{fl/fl} mice. The lower histogram represents the expression levels of CD25 in Foxp3+ cells shown in upper panels. Panel (e) shows flow cytometric analysis of T cell activation markers CD62L and CD44 in CD3+CD4+ Foxp3− (upper panels) and CD3+CD8+ Foxp3− (lower panels) cells in the LNs. Panel (f) shows flow cytometric analysis of cytokine production by splenic CD4+ Foxp3− cells isolated from indicated mice and in vitro stimulated with anti-CD3/CD28 for 5 hrs. Panel (g) shows flow cytometric analysis of IFNγ production by splenic CD8+ Foxp3− cells stimulated with anti-CD3/CD28 for 5 hrs. Data are representative of 5 vs. 5 mice analyzed (d-g). Panel (h) shows histopathology analysis of Foxp3^{Cre}Stat5a/b^{fl/fl} mice. H&E staining of the formalin-fixed tissue sections of the indicated organs of 4-wk-old mice. Scale bar, 100 Representative images of 5 mice analyzed are shown.

FIG. 9, comprising panels (a) through (e), demonstrates rescue of suppressor activity of IL-2Rα-deficient Treg cells upon expression of a constitutively active form of STAT5. Panel (a) shows flow cytometric analysis of Foxp3 and CD25 expression in CD3+CD4+ cells in the LNs and spleens of the indicated mice (4 wk-old). Panel (b) shows flow cytometric analysis of STAT5 phosphorylation in Treg cells. Splenocytes isolated from the indicated mice were stimulated with rmIL-2 (1,000 U/ml) for 20 min, and intracellular levels of tyrosine phosphorylated STAT5 in CD4+YFP+(Foxp3+) cells were analyzed. Panel (c) shows flow cytometric analysis of T cell activation markers CD62L and CD44 in CD3+CD4+ Foxp3− and CD3+CD8+ Foxp3− cells in the LNs of the indicated mice. Panel (d) shows cytokine production by splenic CD4+ Foxp3− cells stimulated for 5 hrs with anti-CD3/CD28. Representative data of three independent experiments are shown (a-d). Panel (e) shows frequency of CD44hi cells among CD3+CD4+ Foxp3− (left graph) and CD3+CD8+ Foxp3− (right graph) cells in the LNs of the indicated mice. Each dot represents a single mouse. Error bars indicate mean+/−S.E.M. Data are representative of two independent experiments.

FIG. 10, comprising panels (a) and (b) demonstrates effects of in vivo IL-2 neutralization on the activation of CD4+ and CD8+ cells. Panel (a) shows representative flow cytometric analyses of LN cells of the indicated mice treated either with IL-2 neutralizing antibody or control IgG. Mice were treated for 2 wks starting from 7 days after birth. Cytokine production by CD4+ Foxp3− and CD8+ Foxp3− cells was analyzed after in vitro stimulation with anti-CD3/CD28 for 5 hrs. Data represent three mice per group analyzed. Panel (b) shows LN cells of Foxp3^{Cre} (upper 6 panels) and Foxp3^{Cre}Il2rb^{fl/fl} (lower 8 panels) mice were unstimulated or stimulated with rmIL-2 (1,000 or 10 U/ml) for 20 min, and intracellular levels of tyrosine phosphorylated STAT5 in Treg (CD4+YFP+CD25hi), Tnaïve (YFP-CD44loCD25lo; CD4+ and CD8+), and Teff (YFP-CD44hi; CD2510 and CD25hi; CD4+ and CD8+) cells were analyzed by flow cytometry. Data are representative of two independent experiments.

FIG. 11, comprising panels (a) through (i), demonstrates characterization of mice harboring Treg cells expressing a constitutively active form of STAT5. Panel (a) shows proliferation of STAT5bCA+ Treg cells after tamoxifen gavage. Three mice were sacrificed and analyzed at each time point. The frequencies of STAT5bCA+ Treg cells among total Treg cells in the spleen were determined by flow cytometry. Error bars indicate +/−S.E.M. Panel (b) shows frequency of STAT5bCA+ Treg cells among total Treg cells in the indicated organs of Foxp3^{Cre-ERT2}ROSA26^{Stat5bCA} mice were determined by flow cytometry three months after a single tamoxifen treatment. Panel (c) shows changes in body weights after tamoxifen gavage. 4-month-old Foxp3Cre-ERT2 (black, n=7) and Foxp3^{Cre-ERT2}ROSA26^{Stat5bCA} (blue, n=7) mice were gavaged with tamoxifen and body weights were monitored the following 4 months. Error bars indicate +/−S.E.M. Panel (d) shows serum chemistry profiles for Foxp3^{Cre-ERT2} (black) and Foxp3^{Cre-ERT2}ROSA26^{Stat5bCA} (blue) mice 4.5 months after tamoxifen gavage. Each dot represents a single mouse. Error bars indicate mean+/−S.E.M. Panel (e) shows TCR Vβ usages of the Treg cells in various tissues were analyzed by flow cytometry 2 months after tamoxifen gavage for Foxp3^{Cre-ERT2}(Cont) and Foxp3^{Cre-ERT2}ROSA26^{Stat5bCA} (CA) mice. MLNs, mesenteric lymph nodes; PPs, Peyer's patches. Representative data of two independent experiments are shown. Panels (f-h) show a general characterization of Treg cells of Foxp3^{Cre-ERT2} (black) and Foxp3^{Cre-ERT2}ROSA26Stat5bCA (blue) mice three months after a single tamoxifen treatment. Panel (f) shows the expression levels of the indicated molecules on Treg cells in the indicated organs. Panel (g) shows frequency of Foxp3+ cells among CD3+CD4+ cells (upper graph) and the expression levels of Foxp3 in the CD3+CD4+ Foxp3+ cells (lower graph) in the indicated organs. Panel (h) shows frequency of Foxp3+ cells among CD3+CD8+ cells in the indicated organs. Each dot represents a single mouse. Error bars indicate mean+/−S.E.M (b, d, f, g, h). Data are representative of two independent experiments (f, g, h). Panel (i) shows increased suppressor activity of STAT5bCA Treg cells. Treg cells were isolated from Foxp3Cre-ERT2 (control) and Foxp3Cre-ERT2ROSA26Stat5bCA (Stat5bCA) mice and co-cultured with T naïve cells (responder cells). The proliferative activity of Treg and responder cells was determined by flow cytometry based on the dilution of CellTrace Violet (CTV) fluorescence intensity. Typical dye dilution patterns of T naïve cells at a 4:1 responder vs. Treg cell ratio are shown in the left two panels. Summary graphs showing the proliferation of co-cultured responder T cells and Treg cells are shown in the right two panels. Note that CTV MFI of cells inversely correlates with cell division. Error bars indicate +/−S.E.M of triplicate wells.

FIG. 12, comprising panels (a) through (e) demonstrates systemic reduction of Teff cell population in the presence of STAT5bCA+ Treg cells. Panels (a) and (b) show frequency of Ki-67+(upper graphs), CD62LhiCD44lo (middle; % Tnaïve), and CD62LloCD44hi (lower; % Teff) cells among CD4+ Foxp3−(a) and CD8+ Foxp3−(b) cells of the indicated organs were determined by flow cytometry. Panel (c) shows splenocytes and mesenteric LN cells of the indicated mice were stimulated with anti-CD3/CD28 for 5 hrs, and the frequencies of the indicated cytokine-producing cells among CD4+ Foxp3− cells were determined by flow cytometry. Panel (d) shows serum Ig levels determined by ELISA. Foxp3^{Cre-ERT2} (black dots) and Foxp3^{Cre-ERT2}ROSA26^{Stat5bCA} (blue dots) mice were analyzed three months after a single tamoxifen treatment (a-d). Panel (e) shows effect of Treg cells expressing a constitutively active form of STAT5 on intestinal carcinogenesis. Foxp3^{Cre-ERT2}Apc^{Min/+} and Foxp3^{Cre-ERT2}ROSA26^{Stat5bCA}Apc^{Min/+} mice were treated with tamoxifen at 4 wk of age and the numbers and sizes of polyps in the distal small intestines were assessed 4 month later using stereomicroscopy. Each dot represents a single mouse. Error bars indicate mean+/−S.E.M (a-e).

FIG. 13, comprising panels (a) through (c), describes RNA-seq analysis performed to acquire data shown in FIG. 5. Panel (a) shows a plot of gene expression (as log₂ normalized read count) in control Tnaïve versus STAT5bCA Tnaïve cells (i.e., naïve CD4+ T cells from Foxp3 ^{Cre-ERT2}ROSA26^{Stat5bCA} mice). The diagonal lines indicate fold change of at least 1.5× or 0.67× fold. Significantly up- and down-regulated genes (defined as genes with at least 1.5× or 0.67× fold change, adjusted P-value≤0.05, and expression above a minimal threshold based on the distribution of all genes) are colored red or blue, respectively, and their numbers are shown. Panel (b) shows a volcano plot showing log₁₀ FDR-adjusted P-values versus logₑ fold change between STAT5bCA and control Treg cells. Genes that fall outside of the x- or y-axis range of this plot are shown on the axes as empty triangles. The vertical and horizontal gray lines indicate 1.5× or 0.67× fold change (±log₂ 1.5=±0.58) and P=0.05 (−log₁₀ 0.05=1.3), respectively. Panel (c) shows network analysis of GO term enrichment among significantly downregulated genes in STAT5bCA expressing vs. control Treg cells. Downregulated genes were analyzed for over-represented GO terms using BiNGO in Cytoscape, and the resulting network was calculated and visualized using EnrichmentMap. Groups of similar GO terms were manually circled. Edge thickness and color are proportional to the similarity coefficient between connected gene sets. Node color is proportional to the FDR-adjusted P-value of the enrichment. Node size is proportional to gene set size.

FIG. 14 shows gene ontology terms enriched among genes up- or down-regulated in STAT5bCA Treg versus control Treg cells.

FIG. 15 demonstrates strategies for generation of a conditional IL2rb allele and IL2rb targeting. The targeting vector was constructed such that upon Cre-mediated deletion, the promoter region and exon 2 which comprises the first ATG of Il2rb were deleted with simultaneous activation of eGFP expression. Shown from top to bottom i) the Il2rb locus with the promoter region, exons and translational start site in exon 2 (E2); ii) the targeting vector comprising an eGFP, a triple SV40 poly A site (tpA), a PGK neopA cassette, a PGK promoter (Pr.) downstream of exon 2, a TK gene, and loxP and frt sites; arrows denote the orientation; iii) the targeted Il2rb locus. Restriction sites, probes used for detection and the expected fragments detected by Southern blot analysis are indicated. Correctly targeted embryonic stem (ES) cell lines were identified by Southern blot analysis of XbaI digested DNA that displayed the 4.0 kb band of the integrated transgene along with the 14.0 kb wild-type band. Co-integration of the 3′ loxP site was verified by PCR analysis using primers that hybridize in a unique region spanning the PGK promoter and the 3′ frt site (forward primer) and in a region upstream of intron 3 of Jl2rb (reverse primer).

FIG. 16 shows a schematic of, and targeting strategy for, ROSA26^{Stat5bCA} allele. The targeting vector was constructed such that CAG promoter driven STAT5bCA is expressed upon Cre-mediated deletion of a STOP cassette. Correctly targeted ES cell lines were identified by Southern blot analysis of EcoRI-digested DNA that displayed the 5.9 kb (probe A; 5′ side) and 11.6 kb (probe F; 3′ side) bands of the integrated trans gene along with the 15.6 kb wild-type band (probe A and F; both sides).

### DEFINITIONS

Administration: As used herein, the term “administration” refers to the administration of a composition to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (including by intratracheal instillation), transdermal, vaginal and vitreal. In some embodiments, administration may be intratumoral or peritumoral. In some embodiments, administration may involve intermittent dosing. In some embodiments, administration may involve continuous dosing (e.g., perfusion) for at least a selected period of time.

Adoptive cell therapy: As used herein, “adoptive cell therapy” or “ACT” involves the transfer of immune cells, e.g Tregs, into subjects. In some embodiments, ACT is a treatment approach that involves the use of lymphocytes with regulatory T-cell activity, the in vitro expansion of these cells to large numbers and their infusion into a subject.

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, nucleic acids (e.g., siRNAs, shRNAs, DNA/RNA hybrids, antisense oligonucleotides, ribozymes), peptides, peptide mimetics, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Amelioration: As used herein, “amelioration” refers to prevention, reduction and/or palliation of a state, or improvement of the state of a subject. Amelioration includes, but does not require, complete recovery or complete prevention of a disease, disorder or condition.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are composed of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are fully human, or are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgG, IgE and IgM, bi- or multi-specific antibodies (e.g., Zybodies®, etc), single chain Fvs, polypeptide-Fc fusions, Fabs, cameloid antibodies, masked antibodies (e.g., Probodies®), Small Modular ImmunoPharmaceuticals (“SMIPs™”), single chain or Tandem diabodies (TandAb®), Anticalins®, Nanobodies®, minibodies, BiTE®s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins®, and a KALBITOR®. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.)).

Antigen: The term “antigen”, as used herein, refers to an agent that elicits an immune response; and/or an agent that binds to a T cell receptor (e.g., when presented by an MEW molecule) or to an antibody or antibody fragment. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer (e.g., other than a nucleic acid or amino acid polymer)) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source), or alternatively may exist on or in a cell. In some embodiments, an antigen is a recombinant antigen.

Antigen presenting cell: The phrase “antigen presenting cell” or “APC,” as used herein, has its art understood meaning referring to cells that process and present antigens to T-cells. Exemplary APC include dendritic cells, macrophages, B cells, certain activated epithelial cells, and other cell types capable of TCR stimulation and appropriate T cell costimulation.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Chimeric antigen receptor: “Chimeric antigen receptor” or “CAR” or “CARs” as used herein refers to engineered receptors, which graft an antigen specificity onto cells (for example T cells such as naïve T cells, central memory T cells, effector memory T cells, regulatory T cells or combination thereof). CARs are also known as artificial T-cell receptors, chimeric T-cell receptors or chimeric immunoreceptors. In some embodiments, CARs comprise an antigen-specific targeting regions, an extracellular domain, a transmembrane domain, one or more co-stimulatory domains, and an intracellular signaling domain.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Constitutively Active: As used herein, the term “constitutively active” refers to a state of elevated and/or more temporally consistent activity as compared with an appropriate reference under comparable conditions. In particular embodiments, a “constitutively active” state is characterized by a consistently detectable level of activity, e.g., above a particular threshold level. In some embodiments, a “constitutively active” state is characterized by presence of an active form of an agent of interest (e.g., of a protein of interest, and/or of a nucleic acid that encodes the protein of interest). In some embodiments, a “constitutively active” state may be achieved through one or more of elevated and/or consistent level of production, inhibited and/or inconsistent level of destruction (e.g., degradation), altered level and/or timing of modification (e.g., to generate or destroy an active form of an agent of interest), etc.

Dosage form: As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

Dosing regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Engineered: Those of ordinary skill in the art, reading the present disclosure, will appreciate that the term “engineered”, as used herein, refers to an aspect of having been manipulated and altered by the hand of man. In particular, the term “engineered cell” refers to a cell that has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation. In some embodiments, a genetic manipulation is or comprises one or more of (i) introduction of a nucleic acid not present in the cell prior to the manipulation (i.e., of a heterologous nucleic acid); (ii) removal of a nucleic acid, or portion thereof, present in the cell prior to the manipulation; and/or (iii) alteration (e.g., by sequence substitution) of a nucleic acid, or portion thereof, present in the cell prior to the manipulation. In some embodiments, a genetic manipulln some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. Those of ordinary skill in the art will appreciate that reference to an “engineered cell” herein may, in some embodiments, encompass both the particular cell to which the manipulation was applied and also any progeny of such cell.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Fusion protein: As used herein, the term “fusion protein” generally refers to a polypeptide including at least two segments, each of which shows a high degree of amino acid identity to a peptide moiety that (1) occurs in nature, and/or (2) represents a functional domain of a polypeptide. Typically, a polypeptide containing at least two such segments is considered to be a fusion protein if the two segments are moieties that (1) are not included in nature in the same peptide, and/or (2) have not previously been linked to one another in a single polypeptide, and/or (3) have been linked to one another through action of the hand of man.

Gene: As used herein, the term “gene” has its meaning as understood in the art. It will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs, RNAi-inducing agents, etc. For the purpose of clarity we note that, as used in the present application, the term “gene” generally refers to a portion of a nucleic acid that encodes a protein; the term may optionally encompass regulatory sequences, as will be clear from context to those of ordinary skill in the art. This definition is not intended to exclude application of the term “gene” to non-protein—coding expression units but rather to clarify that, in most cases, the term as used in this document refers to a protein-coding nucleic acid.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Heterologous: As used herein, the term “heterologous” refers to an agent (e.g. a nucleic acid, protein, cell, tissue, etc) that is present in a particular context as a result of engineering as described herein (i.e., by application of a manipulation to the context). To give but a few examples, a nucleic acid or protein that is ordinarily or naturally found in a first cell type and not in a second cell type (e.g., in a bacterial cell and not in a mammalian cell, in a cell from a first tissue and not in a cell from a second tissue, in a cell of a first microbial species but not in a cell of a second microbial species, etc) may be “heterologous” to the second cell type. Analogously, a cell or tissue that is ordinarily or naturally found in a first organism and not in a second organism (e.g., in a rodent and not in a mammal, etc) may be “heterologous” to the second organism. Those of ordinary skill in the art will understand the scope and content of the term “heterologous” as used herein.

Immune response: As used herein, the term “immune response” refers to a response elicited in an animal. In some embodiments, an immune response may refer to cellular immunity, humoral immunity or may involve both. In some embodiments, an immune response may be limited to a part of the immune system. For example, in certain embodiments, an immune response may be or comprise an increased IFNγ response. In certain embodiments, immune response may be or comprise mucosal IgA response (e.g., as measured in nasal and/or rectal washes). In certain embodiments, an immune response may be or comprise a systemic IgG response (e.g., as measured in serum). In certain embodiments, an immune response may be or comprise a neutralizing antibody response. In certain embodiments, an immune response may be or comprise a cytolytic (CTL) response by T cells. In certain embodiments, an immune response may be or comprise reduction in immune cell activity.

Improve, increase, or reduce: As used herein, the terms “improve,” “increase” or “reduce,” or grammatical equivalents, indicate values that are relative to an appropriate reference measurement, as will be understood by those of ordinary skill in the art. To give but a few examples, in some embodiments, application of such a term in reference to an individual who has received a particular treatment may indicate a change relative to a comparable individual who has not received the treatment, and/or to the relevant individual him/herself prior to administration of the treatment, etc.

Individual, subject: As used herein, the terms “subject” or “individual” refer to a particular human or non-human mammalian organism; in many embodiments, the terms refer to a human. In some embodiments, an “individual” or “subject” may be a member of a particular age group (e.g., may be a fetus, infant, child, adolescent, adult, or senior). In some embodiments, an “individual” or “subject” may be suffering from or susceptible to a particular disease, disorder or condition (i.e., may be a “patient”).

Nucleic acid: As used herein, “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments a nucleic acid has a nucleotide sequence comprising at least one element that encodes, or is the complement of a sequence that encodes, a polypeptide. In some embodiments, a nucleic acid has enzymatic activity.

Operably linked: As used herein, “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest. The term “expression control sequence” as used herein refers to polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism. For example, in prokaryotes, such control sequences generally include promoter, ribosomal binding site, and transcription termination sequence, while in eukaryotes, typically, such control sequences include promoters and transcription termination sequence. The term “control sequences” is intended to include components whose presence is essential for expression and processing, and can also include additional components whose presence is advantageous, for example, leader sequences and fusion partner sequences.

Patient: As used herein, the term “patient” refers to a organism who is suffering from or susceptible to a disease, disorder or condition and/or who will receive administration of a diagnostic, prophylactic, and/or therapeutic regimen. In many embodiments, a patient displays one or more symptoms of a disease, disorder or condition. In some embodiments, a patient has been diagnosed with one or more diseases, disorders or conditions. In some embodiments, the disorder or condition is or includes cancer, or presence of one or more tumors. In some embodiments, a patient is receiving or has received certain therapy to diagnose, prevent (i.e., delay onset and/or frequency of one or more symptoms of) and/or to treat a disease, disorder, or condition.

Peptide: The term “peptide” as used herein refers to a polypeptide that is typically relatively short, for example having a length of less than about 100 amino acids, less than about 50 amino acids, less than 20 amino acids, or less than 10 amino acids.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Protein: As used herein, the term “protein”, refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof.

Reference: As used herein, “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Suffering from: An individual who is “suffering from” a disease, disorder, or condition (e.g., cancer) has been diagnosed with and/or exhibits one or more symptoms of the disease, disorder, or condition.

Symptoms are reduced: According to the present invention, “symptoms are reduced” when one or more symptoms of a particular disease, disorder or condition is reduced in magnitude (e.g., intensity, severity, etc.) or frequency. For purposes of clarity, a delay in the onset of a particular symptom is considered one form of reducing the frequency of that symptom. It is not intended that the present invention be limited only to cases where the symptoms are eliminated. The present invention specifically contemplates treatment such that one or more symptoms is/are reduced (and the condition of the subject is thereby “improved”), albeit not completely eliminated.

T cell receptor: The terms “T cell receptor” or “TCR” are used herein in accordance with the typical understanding in the field, in reference to antigen-recognition molecules present on the surface of T-cells. During normal T-cell development, each of the four TCR genes, α, β, γ, and δ, can rearrange, so that T cells of a particular individual typically express a highly diverse population of TCR proteins.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically effective amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. For example, in some embodiments, “therapeutically effective amount” refers to an amount which, when administered to an individual in need thereof in the context of inventive therapy, will block, stabilize, attenuate, or reverse a cancer-supportive process occurring in said individual, or will enhance or increase a cancer-suppressive process in said individual. In the context of cancer treatment, a “therapeutically effective amount” is an amount which, when administered to an individual diagnosed with a cancer, will prevent, stabilize, inhibit, or reduce the further development of cancer in the individual. A particularly preferred “therapeutically effective amount” of a composition described herein reverses (in a therapeutic treatment) the development of a malignancy such as a pancreatic carcinoma or helps achieve or prolong remission of a malignancy. A therapeutically effective amount administered to an individual to treat a cancer in that individual may be the same or different from a therapeutically effective amount administered to promote remission or inhibit metastasis. As with most cancer therapies, the therapeutic methods described herein are not to be interpreted as, restricted to, or otherwise limited to a “cure” for cancer; rather the methods of treatment are directed to the use of the described compositions to “treat” a cancer, i.e., to effect a desirable or beneficial change in the health of an individual who has cancer. Such benefits are recognized by skilled healthcare providers in the field of oncology and include, but are not limited to, a stabilization of patient condition, a decrease in tumor size (tumor regression), an improvement in vital functions (e.g., improved function of cancerous tissues or organs), a decrease or inhibition of further metastasis, a decrease in opportunistic infections, an increased survivability, a decrease in pain, improved motor function, improved cognitive function, improved feeling of energy (vitality, decreased malaise), improved feeling of well-being, restoration of normal appetite, restoration of healthy weight gain, and combinations thereof. In addition, regression of a particular tumor in an individual (e.g., as the result of treatments described herein) may also be assessed by taking samples of cancer cells from the site of a tumor such as a pancreatic adenocarcinoma (e.g., over the course of treatment) and testing the cancer cells for the level of metabolic and signaling markers to monitor the status of the cancer cells to verify at the molecular level the regression of the cancer cells to a less malignant phenotype. For example, tumor regression induced by employing the methods of this invention would be indicated by finding a decrease in one or more pro-angiogenic markers, an increase in anti-angiogenic markers, the normalization (i.e., alteration toward a state found in normal individuals not suffering from cancer) of metabolic pathways, intercellular signaling pathways, or intracellular signaling pathways that exhibit abnormal activity in individuals diagnosed with cancer. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Transformation: As used herein, “transformation” refers to any process by which exogenous DNA is introduced into a host cell. Transformation may occur under natural or artificial conditions using various methods well known in the art. Transformation may rely on any known method for the insertion of foreign nucleic acid sequences into a prokaryotic or eukaryotic host cell. In some embodiments, a particular transformation methodology is selected based on the host cell being transformed and may include, but is not limited to, viral infection, electroporation, mating, lipofection. In some embodiments, a “transformed” cell is stably transformed in that the inserted DNA is capable of replication either as an autonomously replicating plasmid or as part of the host chromosome. In some embodiments, a transformed cell transiently expresses introduced nucleic acid for limited periods of time.

Treatment: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### DETAILED DESCRIPTION OF CERTAIN EMBODIMENTS

The present invention provides, among other things, compositions and methods relating to modified regulatory T-cells (Treg) and their use in the treatment of various diseases, disorders, and conditions. Specifically, the present invention contemplates the use of engineered Tregs for the treatment of autoimmune and/or inflammatory diseases.

### Regulatory T Cells

Regulatory T cells (Treg) are important in maintaining homeostasis, controlling the magnitude and duration of the inflammatory response, and in preventing autoimmune and allergic responses.

The Forkhead box P3 transcription factor (Foxp3) has been shown to be a key regulator in the differentiation and activity of Treg. In fact, loss-of-function mutations in the Foxp3 gene have been shown to lead to the lethal IPEX syndrome (immune dysregulation, polyendocrinopathy, enteropathy, X-linked). Patients with IPEX suffer from severe autoimmune responses, persistent eczema, and colitis. Regulatory T (Treg) cells expressing transcription factor Foxp3 play a key role in limiting inflammatory responses in the intestine (Josefowicz, S. Z. et al. Nature, 2012, 482, 395-U1510).

In general Tregs are thought to be mainly involved in suppressing immune responses, functioning in part as a “self-check” for the immune system to prevent excessive reactions. In particular, Tregs are involved in maintaining tolerance to self-antigens, harmless agents such as pollen or food, and abrogating autoimmune disease.

Tregs are found throughout the body including, without limitation, the gut, skin, lung, and liver. Additionally, Treg cells may also be found in certain compartments of the body that are not directly exposed to the external environment such as the spleen, lymph nodes, and even adipose tissue. Each of these Treg cell populations is known or suspected to have one or more unique features and additional information may be found in Lehtimaki and Lahesmaa, Regulatory T cells control immune responses through their non-redundant tissue specific features, 2013, FRONTIERS IN IMMUNOL., 4(294): 1-10, the disclosure of which is hereby incorporated in its entirety.

Typically, Tregs are known to require TGF-β and IL-2 for proper activation and development. Tregs, expressing abundant amounts of the IL-2 receptor (IL-2R), are reliant on IL-2 produced by activated T cells. Tregs are known to produce both IL-10 and TGF-β, both potent immune suppressive cytokines. Additionally, Tregs are known to inhibit the ability of antigen presenting cells (APCs) to stimulate T cells. One proposed mechanism for APC inhibition is via CTLA-4, which is expressed by Foxp3⁺ Treg. It is thought that CTLA-4 may bind to B7 molecules on APCs and either block these molecules or remove them by causing internalization resulting in reduced availability of B7 and an inability to provide adequate co-stimulation for immune responses. Additional discussion regarding the origin, differentiation and function of Treg may be found in Dhamne et al., Peripheral and thymic Foxp3+ regulatory T cells in search of origin, distinction, and function, 2013, Frontiers in Immunol., 4 (253): 1-11, the disclosure of which is hereby incorporated in its entirety.

### STAT

Members of the signal transducer and activator of transcription (STAT) protein family are intracellular transcription factors that mediate many aspects of cellular immunity, proliferation, apoptosis and differentiation. They are primarily activated by membrane receptor-associated Janus kinases (JAK). Dysregulation of this pathway is frequently observed in primary tumors and leads to increased angiogenesis, enhanced survival of tumors and immunosuppression. Gene knockout studies have provided evidence that STAT proteins are involved in the development and function of the immune system and play a role in maintaining immune tolerance and tumor surveillance.

There are seven mammalian STAT family members that have been identified: STAT1, STAT2, STAT3, STAT4, STAT5 (including STAT5A and STAT5B), and STATE.

Extracellular binding of cytokines or growth factors induce activation of receptor-associated Janus kinases, which phosphorylate a specific tyrosine residue within the STAT protein promoting dimerization via their SH2 domains. The phosphorylated dimer is then actively transported to the nucleus via an importin α/β ternary complex. Originally, STAT proteins were described as latent cytoplasmic transcription factors as phosphorylation was thought to be required for nuclear retention. However, unphosphorylated STAT proteins also shuttle between the cytosol and nucleus, and play a role in gene expression. Once STAT reaches the nucleus, it binds to consensus a DNA-recognition motif called gamma-activated sites (GAS) in the promoter region of cytokine-inducible genes and activates transcription. The STAT protein can be dephosphorylated by nuclear phosphatases, which leads to inactivation of STAT and subsequent transport out of the nucleus by a exportin-RanGTP complex.

In some embodiments, a STAT protein of the present disclosure may be a STAT protein that comprises a modification that modulates its expression level or activity. In some embodiments such modifications include, among other things, mutations that effect STAT dimerization, STAT protein binding to signaling partners, STAT protein localization or STAT protein degradation. In some embodiments, a STAT protein of the present disclosure is constitutively active. In some embodiments, a STAT protein of the present disclosure is constitutively active due to constitutive dimerization. In some embodiments, a STAT protein of the present disclosure is constitutively active due to constitutive phosphorylation as described in Onishi, M. et al., Mol. Cell. Biol. July 1998 vol. 18 no. 7 3871-3879 the entirety of which is herein incorporated by reference.

### Cell Engineering

Those skilled in the art are aware of a wide variety of technologies available for engineering of cells (e.g., mammalian cells, and particularly mammalian Treg cells). For example, various systems for introducing nucleic acids for expression in and/or integration into such cells are well known in the art, as are various strategies for achieving epigenetic modification of cells.

In some embodiments, cell engineering technologies appropriate for use in accordance with the present disclosure may be or comprise introduction of one or more heterologous nucleic acids into a cell. In some embodiments, technologies for introduction of a heterologous nucleic acid into a cell include, among other things, transfection, electroporation including nucleofection, and transduction. Various vector systems for introduction of heterologous nucleic acids are known in the art, including but not limited to, plasmids, bacterial artificial chromosomes, yeast artificial chromosomes, and viral systems (e.g, adenoviruses and lentiviruses).

In some embodiments, cell engineering technologies appropriate for use in accordance with the present disclosure may be or comprise introduction of one or more heterologous proteins into a cell. In some embodiments, technologies for introduction of a heterologous protein into a cell include, among other things, transfection, transduction with cell permeable peptides (e.g. TAT), and nanoparticle delivery.

In general, cells may be engineered as described herein so that they express a constitutively active STAT protein (i.e., so that level and/or activity of an active form of a STAT protein is constitutively present in the cell). Those of ordinary skill in the art will appreciate that a variety of engineering strategies could achieve such constitutively active expression. For example, to name but a few, in some embodiments, a STAT protein variant may be introduced; a protein inducing the expression of STAT may be introduced, a protein increasing the stability of STAT protein may be introduced, or a protein reducing the degradation of STAT may be introduced.

In some embodiments, a introduced nucleic acid may be or comprise a sequence that encodes, or is complimentary to a nucleic acid that encodes, part or all of a STAT protein. In some embodiments, a introduced nucleic acid may be or comprise a sequence that encodes, or is complimentary to a nucleic acid that encodes, part or all of a STAT protein that is constitutively expressed.

In some embodiments, an introduced nucleic acid may be or comprise a regulatory sequence functional in the cell to regulate expression of a nucleic acid that encodes, or is complimentary to a nucleic acid that encodes, part or all of a STAT protein.

In some embodiments, an introduced nucleic acid may be or comprise a sequence that encodes, or is complimentary to a nucleic acid that encodes, a constitutively active STAT protein. In some embodiments, an introduced protein may be or comprise a constitutively active STAT protein.

In some embodiments, the methods and compositions of the present disclosure relate to the use of a subjects own, or autologous, cells. In some embodiments, the methods and compositions of the present disclosure relate to the use of heterologous cells.

Chimeric antigen receptor T-cells (CAR-T) are among the methods of treatment using engineered T-cells that are being developed. CAR T-cells are T-cells engineered to express an exogenous antigen receptor. Such antigen receptors are referred to as chimeric because they are composed of domains from different proteins. In some embodiments the portions of a CAR can include, among other things, an antigen recognition domain, a transmembrane domain, and a cytoplasmic domain.

As much of the effort in disease directed cell engineering and CAR-T cell development is focused on destruction of tumors or infected cells the primary focus in the art has been on the modification of cytolytic T-cells (CD8+). Those skilled in the art are aware that current adoptive cell therapy regimens with CAR-T cells comprises the co-administration of CAR-T cells with IL-2.

In contrast, the methods and compositions of the present disclosure contemplate an adoptive cell therapy regimen without the need for co-administration with IL-2. Alternatively, the methods and compositions of the present disclosure contemplate an adoptive cell therapy regimen with co-administration with IL-2. The methods and compositions of the present disclosure are relevant to the engineering Treg cells for the treatment of various diseases, disorders and conditions.

### Diseases, Disorders, and Conditions

In some embodiments, methods and compositions of the present disclosure are relevant to the treatment of, among other things, diseases, disorders or conditions characterized by inflammation. In some embodiments, methods and compositions of the present disclosure are relevant to the treatment of, among other things, diseases, disorders or conditions characterized by autoimmunity. In some embodiments, methods and compositions of the present disclosure are relevant to the treatment of inflammation and/or autoimmune disorders affecting the gastrointestinal tract. In some embodiments, methods and compositions of the present disclosure are relevant to the treatment of inflammation and/or autoimmune disorders affecting the nervous system.

Inflammation

Inflammation, as used herein, refers to the localized protective response of vascular tissues to injury, irritation or infection. Inflammatory conditions are characterized by one or more of the following symptoms: redness, swelling, pain and loss of function. Inflammation is a protective attempt by the organism to remove the harmful stimuli and begin the healing process. Although infection is caused by a microorganism, inflammation is one of the responses of the organism to the pathogen.

Inflammation can be classified as either acute or chronic. Acute inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process.

Inflammation may be caused by a number of agents, including infectious pathogens, toxins, chemical irritants, physical injury, hypersensitive immune reactions, radiation, foreign irritants (dirt, debris, etc.), frostbite, and burns. Transplanted or transfused tissues, organs or blood products, among other things, can also be included in the broad category of foreign irritants. Graft versus host disease is one example of a disease, disorder, or condition arising from inflammation from transplanted or transfused tissues, organs or blood products. Types of inflammation include colitis, bursitis, appendicitis, dermatitis, cystitis, rhinitis, tendonitis, tonsillitis, vasculitis, and phlebitis.

Autoimmunity

Autoimmunity refers to the presence of a self-reactive immune response (e.g., auto-antibodies, self-reactive T-cells). Autoimmune diseases, disorders, or conditions arise from autoimmunity through damage or a pathologic state arising from an abnormal immune response of the body against substances and tissues normally present in the body. Damage or pathology as a result of autoimmunity can manifest as, among other things, damage to or destruction of tissues, altered organ growth, and/or altered organ function.

Types of autoimmune diseases, disorders or conditions include type I diabetes, alopecia areata, vasculitis, temporal arteritis, rheumatoid arthritis, lupus, celiac disease, Sjogrens syndrome, polymyalgia rheumatica, and multiple sclerosis.

### Administration

Certain embodiments of the disclosure include administration of an engineered regulatory T-cell to a subject; or a composition comprising of an engineered regulatory T-cell. In some embodiments, a regulatory T-cell is obtained from a subject and modified as described herein to obtain an engineered regulatory T-cell. Thus, in some embodiments, an engineered regulatory T-cell comprises an autologous cell that is administered into the same subject from which an immune cell was obtained. Alternatively, an immune cell is obtained from a subject and is transformed, e.g., transduced, as described herein, to obtain an engineered regulatory T-cell that is allogenically transferred into another subject.

In some embodiments, a regulatory T-cell for use in accordance with the present disclosure is obtained by collecting a sample from a subject containing immune cells and isolating regulatory T-cells from the sample. In some embodiments, a regulatory T-cell for use in accordance with the present disclosure is obtained by collecting a sample from a subject containing immune cells and isolating an immune cell sub-population (e.g. CD4+ cells, CD8+ cells, etc.) for use in in vitro generation of regulatory T-cells. In some embodiments, a regulatory T-cell for use in accordance with the present disclosure is obtained by collecting a sample from a subject containing immune cells and isolating naïve CD4+ T-cells for use in for in vitro generation of regulatory T-cells. In some embodiments, a regulatory T-cell for use in accordance with the present disclosure is obtained by collecting a sample from a subject containing immune cells and isolating naïve CD8+ T-cells for use in for in vitro generation of regulatory T-cells.

Those skilled in the art are aware of a wide variety of techniques available for in vitro generation of regulatory T-cell. For example, activation of isolated immune cells with plate-bound anti-CD3 and soluble anti-CD28 in the presence of TGF-β.

In some embodiments, an engineered regulatory T-cell is autologous to a subject, and the subject can be immunologically naïve, immunized, diseased, or in another condition prior to isolation of an immune cell from the subject.

In some embodiments, additional steps can be performed prior to administration of an engineered regulatory T-cell to a subject. For instance, an engineered regulatory T-cell can be expanded in vitro after modification, e.g. introduction of a chimeric antigen receptor and/or modified STAT protein, but prior to the administration to a subject. In vitro expansion can proceed for 1 day or more, e.g., 2 days or more, 3 days or more, 4 days or more, 6 days or more, or 8 days or more, prior to the administration to a subject. Alternatively, or in addition, in vitro expansion can proceed for 21 days or less, e.g., 18 days or less, 16 days or less, 14 days or less, 10 days or less, 7 days or less, or 5 days or less, prior to administration to a subject. For example, in vitro expansion can proceed for 1-7 days, 2-10 days, 3-5 days, or 8-14 days prior to the administration to a subject.

In some embodiments, during in vitro expansion, an engineered regulatory T-cell can be stimulated with an antigen (e.g., a TCR antigen). Antigen specific expansion optionally can be supplemented with expansion under conditions that non-specifically stimulate lymphocyte proliferation such as, for example, anti-CD3 antibody, anti-Tac antibody, anti-CD28 antibody, or phytohemagglutinin (PHA). The expanded engineered regulatory T-cell can be directly administered into a subject or can be frozen for future use, i.e., for subsequent administrations to a subject.

In certain embodiments, an engineered regulatory T-cell is administered prior to, substantially simultaneously with, or after the administration of another therapeutic agent. An engineered regulatory T-cell described herein can be formed as a composition, e.g., a an engineered regulatory T-cell and a pharmaceutically acceptable carrier. In certain embodiments, a composition is a pharmaceutical composition comprising at least one engineered regulatory T-cell described herein and a pharmaceutically acceptable carrier, diluent, and/or excipient. Pharmaceutically acceptable carriers described herein, for example, vehicles, adjuvants, excipients, and diluents, are well-known and readily available to those skilled in the art. Preferably, the pharmaceutically acceptable carrier is chemically inert to the active agent(s), e.g., an engineered regulatory T-cell, and does not elicit any detrimental side effects or toxicity under the conditions of use.

A composition can be formulated for administration by any suitable route, such as, for example, intravenous, intratumoral, intraarterial, intramuscular, intraperitoneal, intrathecal, epidural, and/or subcutaneous administration routes. Preferably, the composition is formulated for a parenteral route of administration.

A composition suitable for parenteral administration can be an aqueous or nonaqueous, isotonic sterile injection solution, which can contain anti-oxidants, buffers, bacteriostats, and solutes, for example, that render the composition isotonic with the blood of the intended recipient. An aqueous or nonaqueous sterile suspension can contain one or more suspending agents, solubilizers, thickening agents, stabilizers, and preservatives.

Dosage administered to a subject, particularly a human, will vary with the particular embodiment, the composition employed, the method of administration, and the particular site and subject being treated. However, a dose should be sufficient to provide a therapeutic response. A clinician skilled in the art can determine the therapeutically effective amount of a composition to be administered to a human or other subject in order to treat or prevent a particular medical condition. The precise amount of the composition required to be therapeutically effective will depend upon numerous factors, e.g., such as the specific activity of the engineered regulatory T-cell, and the route of administration, in addition to many subject-specific considerations, which are within those of skill in the art.

Any suitable number of engineered regulatory T-cells can be administered to a subject. While a single engineered regulatory T-cell described herein is capable of expanding and providing a therapeutic benefit, in some embodiments, 10² or more, e.g., 10³ or more, 10⁴ or more, 10⁵ or more, or 10⁸ or more, engineered regulatory T-cells are administered. Alternatively, or additionally 10¹² or less, e.g., 10¹¹ or less, 10⁹ or less, 10⁷ or less, or 10⁵ or less, engineered regulatory T-cells described herein are administered to a subject. In some embodiments, 10²-10⁵, 10⁴-10⁷, 10³-10⁹, or 10⁵-10¹⁰ engineered regulatory T-cells described herein are administered.

A dose of an engineered regulatory T-cell described herein can be administered to a mammal at one time or in a series of subdoses administered over a suitable period of time, e.g., on a daily, semi-weekly, weekly, bi-weekly, semi-monthly, bi-monthly, semi-annual, or annual basis, as needed. A dosage unit comprising an effective amount of an engineered regulatory T-cell may be administered in a single daily dose, or the total daily dosage may be administered in two, three, four, or more divided doses administered daily, as needed.

Route of administration can be parenteral, for example, administration by injection, transnasal administration, transpulmonary administration, or transcutaneous administration. Administration can be systemic or local by intravenous injection, intramuscular injection, intraperitoneal injection, subcutaneous injection.

### EXEMPLIFICATION

### Example 1: Materials and Methods

The present Example describes the materials and methods used in Example 2. Mice.

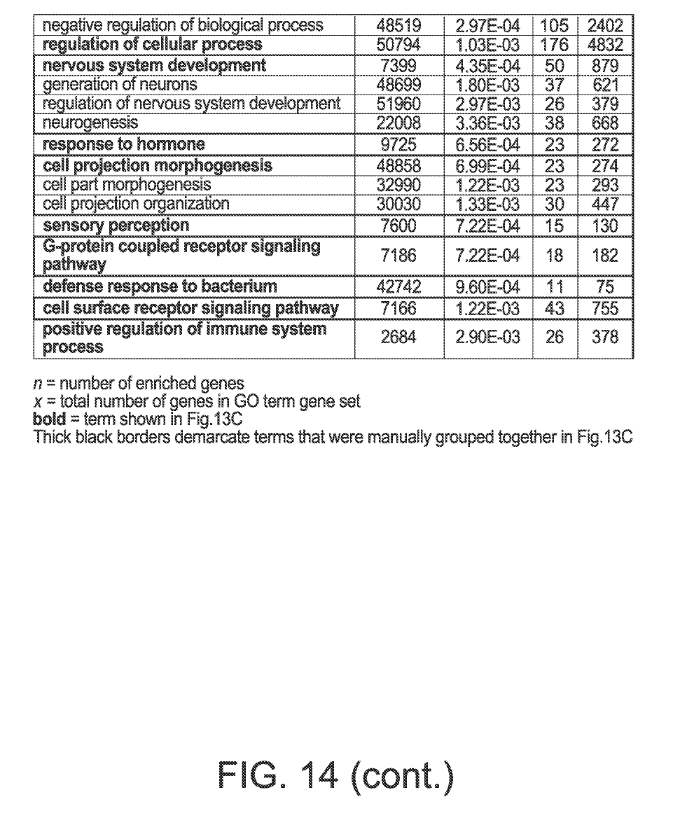

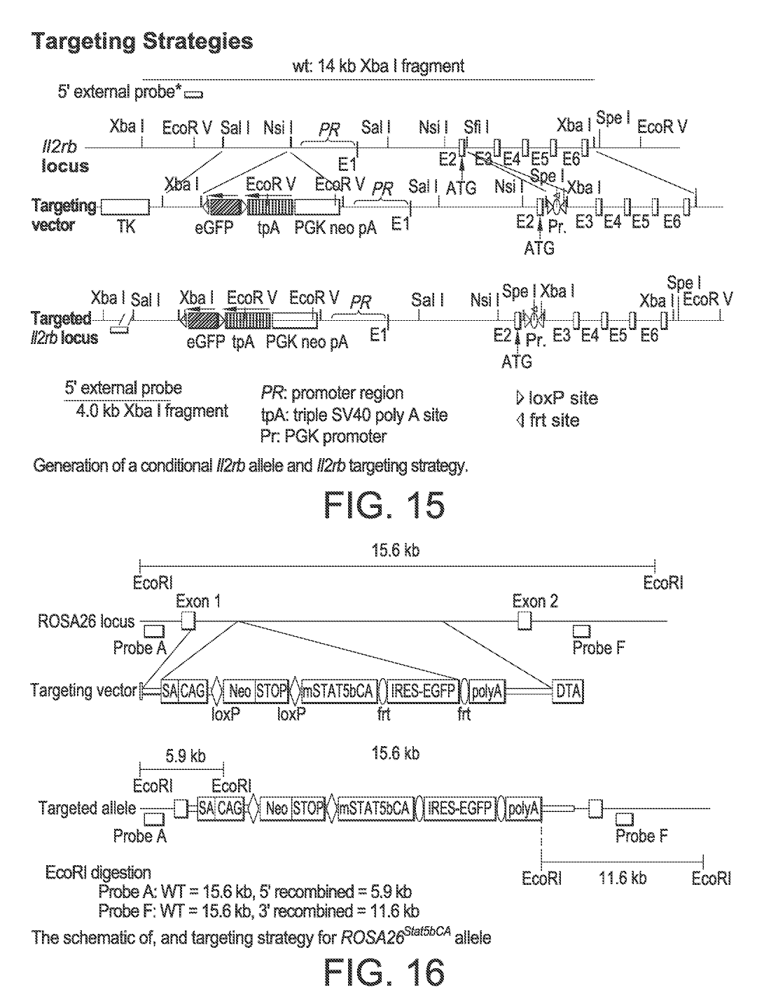

Foxp3^{Cre} and Foxp3^{Cre-ERT2} mice were described previously^{16,43}. Il2ra^{fl} mice were kind gift from Biogen. Stat5a/b^{fl} mice were provided by Lothar Henninghausen (NIH). ApcMin mice were purchased from the Jackson Laboratory. The targeting strategies for Il2rb^{fl} (generated by Ulf Klein) and ROSA26^{Stat5bCA} alleles are shown in FIGS. 15 and 16. The backbone of the targeting vector for ROSA26 locus was kindly provided by Dr. Klaus Rajewsky (Harvard Medical School). The vector encoding murine STAT5bCA was kindly provided by Dr. Toshio Kitamura (the University of Tokyo). Tcra^{fl} mice were described previously³⁴. The experimental mice were either generated on or backcrossed onto a C57BL/6 (B6) background, bred and housed in the specific pathogen-free animal facility at Memorial Sloan Kettering Cancer Center and were used in accordance with institutional guidelines. For survival analysis, mice were monitored daily and unhealthy mice were euthanized once they are found lethargic and counted as non-survivors. For tamoxifen treatment, tamoxifen (Sigma-Aldrich) was dissolved in olive oil at a concentration of 40 mg/ml. Mice were given oral gavage of 100 μl of tamoxifen emulsion per treatment. In EAE and infection experiments, mice were challenged 2 to 3 months after a single tamoxifen gavage and assessed as described previously³⁷.

### Flow Cytometry and Cell Sorting.

Cells were stained with fluorescently tagged antibodies purchased from eBioscience, BD Biosciences, Tonbo Bioscience, or R&D Systems and analyzed using a BD LSR II flow cytometer. Flow cytometry data were analyzed using FlowJo software (TreeStar). For intracellular cytokine staining, cells were stimulated for 5 hrs with CD3 and CD28 antibodies (5 μg/ml each) in the presence of brefeldin A or monensin, harvested and stained with eBioscience Fixation Permeabilization kit. For intracellular tyrosine-phosphorylated STAT5 staining, cells were stimulated with or without rmIL-2 for 20 min, fixed and permeabilized with 4% PFA followed by 90% methanol, and stained with anti-PY-STAT5 antibody (BD Biosciences). Cell sorting of Foxp3+ and Foxp3− cells was performed based on YFP or GFP expression using a BD FACSAria II cell sorter. The following monoclonal antibodies were used for flow cytometry: B220 (RA3-6B2), CD103 (2E7), CD11b (M1/70), CD11c (N418), CD122 (5H4), CD127 (A7R34), CD132 (TUGm2), CD25 (PC61), CD3 (17A2), CD4 (RM4-5), CD44 (IM7), CD45 (30-F11), CD62L (MEL-14), CD69 (H1.2F3), CD8 (5H10), CD80 (16-10A1), CD86 (GL1), CTLA-4 (UC10-4B9), Foxp3 (FJK-16s), GITR (DTA-1), Gr-1 (RB6-8C5), IFNγ (XMG1.2), IL-13 (eBio13A), IL-17 (eBio17B7), IL-4 (11B11), Ki-67 (B56), KLRG1 (2F1), MHC class II (M5/114.15.2), PY-STAT5 (47/Stat5/pY694), TCRβ (H57-597), TNFα (MP6-XT22), Vβ10b (B21.5), Vβ11 (RR3-15), Vβ12 (MR11-1), Vβ13 (MR12-3), Vβ14 (14-2), Vβ2 (B20.6), Vβ3 (KJ25), Vβ4 (KT4), Vβ5.1/5.2 (MR9-4), Vβ6 (RR4-7), Vβ7 (TR310), Vβ8.1/8.2 (MR5-2), Vβ8.3 (1B3.3), Vβ9 (MR10-2).

### Listeria and Vaccinia Infection.

Mice were intravenously injected into the tail vein with Listeria monocytogenes (LM10403S; 2000 cells/mouse) on day 0 and analyzed on day 8. For the detection of Listeria-specific immune responses, splenic DCs from unchallenged B6 mice sorted using CD11c microbeads (Miltenyi) were cultured in wells of a 96 well U-bottom plate (2×10⁴ cells/well) with heat-killed Listeria monocytogenes (2×10⁷ cells/well) for 6 hr prior to the analysis. The cells were then co-cultured with splenic T cells obtained from Listeria-infected mice (1×10⁵ cells/well) for 5 hrs in the presence of brefeldin A, and cytokine producing T cells were detected by flow cytometry. For vaccinia virus infection, mice were intraperitoneally injected with non-replicating virus (5×10⁷ PFU/mouse) on day 0 and analyzed on day 8. Splenocytes were re-stimulated with several vaccinia virus derived antigenic peptides (1 μg/ml) for 5 hrs in the presence of brefeldin A, and cytokine producing T cells were detected by flow cytometry.

### In Vivo IL-2 Neutralization.

Mice were i.p. injected with a cocktail of two different anti-IL-2 monoclonal antibodies JES6-1 and S4B6-1 (BioXcell) or isotype matched control antibody (rat IgG2a, 2A3; BioXcell), 200 μg each, twice a week, starting from 7 days after birth.

### TAT-Cre Protein Treatment of T Cells.

For the induction of STAT5bCA expression in non-Treg cells, 1×10⁷ CD4+ Foxp3− or CD8+ Foxp3− T cells sorted from the LNs and spleens of Foxp3^{Cre}ROSA26^{Stat5bCA} mice were resuspended in 2 ml of serum-free RPMI media containing a TAT-Cre recombinase (Millipore; 50 μg/ml) and incubated at 37° C. for 45 min. The cells were washed with RPMI containing 10% FCS, resuspended in PBS, and injected into T cell-deficient (Tcrb−/−Tcrd−/−) mice together with or without separately sorted Treg cells for in vivo suppression assay.

### In Vitro IL-2 Capture Assay.

Pooled cells from LNs and spleens were depleted of B cells and accessary cells by panning and T cells were enriched. The cells were stained with anti-CD8 and anti-B220 Abs, and CD4+ Treg cells were sorted on the basis of GFP (YFP) expression alone in CD8-negative population. The sorted cells were divided among 8 wells of a 96-well V-bottomed plate (2×10⁵ cells/well) in 25 μl RPMI medium (10% FCS) with or without increasing doses of recombinant human IL-2 (0.016 to 12 U/ml), followed by incubation for 2 h at 37° C. Depletion of IL-2 from the medium was assessed with the BD Cytometric Bead Array and Human IL-2 Enhanced Sensitivity Flex Set according to the manufacturer's instructions (BD Biosciences).

### In Vitro T-DC Conjugation Assay.

Treg cells and non-Treg cells were sorted in the same manner as IL-2 capture assay. Splenic CD11c+ DCs were isolated by MACS from B6 mice injected with Flt3L-secreting B16 melanoma cells. Treg and non-Treg cells were stained with CFSE. DCs were stained with CellTrace Violet (Molecular Probes). 1×10⁴ Treg or non-Treg cells were cultured together with graded numbers of DCs (1×104 to 1×105) in a 96-well round-bottomed plate for 720 min in the presence or absence of rmIL-2 (100 IU/ml). Frequencies of Treg cells conjugated with DCs (% CTV+CFSE+/CFSE+) were analyzed by FACS.

### In Vitro Suppression Assay.

Naïve CD4+ T cells (responder cells) and Treg cells were FACS purified and stained with CellTrace Violet (CTV). 4×10⁴ naïve CD4+ T cells were cultured with graded numbers of Treg cells in the presence of 1×10⁵ irradiated, T-cell-depleted, CF SE-stained splenocytes and 1 μg/ml anti-CD3 antibody in a 96 round-bottom plate for 80 hrs. Cell proliferation of responder T cells and Treg cells (live CFSE-CD4+ Foxp3− and Foxp3+) was determined by flow cytometry based on the dilution of fluorescence intensity of CTV of the gated cells

### Measurements of Serum and Fecal Immunoglobulin Levels.

Serum IgM, IgG1, IgG2a, IgG2b, IgG2c, IgG3 and IgA levels were determined by ELISA using SBA Clonotyping System (Southern Biotech). IgE ELISA was performed using biotinylated anti-IgE antibody (BD Biosciences) and HRP-conjugated streptavidin. For measurement of fecal IgA levels, fresh fecal pellets were collected and dissolved in extraction buffer (7 μl per mg pellet) containing 50 mM Tris-HCl, 150 mM NaCl, 0.5% NP-40, 1 mM EDTA, 1 mM DTT, and protease inhibitor cocktail (Complete mini; Roche). Supernatants were collected after centrifugation, titrated, and IgA levels were measured by ELISA.

### Statistical Analysis for Animal Experiments

Statistical analyses were performed using Prism software with two-tailed unpaired Student's t test. Welch's correction was applied when F test was positive. P values<0.05 were considered significant. *, P<0.05; **, P<0.01; ***, P<0.001; NS, not significant.

### RNA Sequencing.

Male 8-wk-old Foxp3^{Cre-ERT2}ROSA26^{Stat5bCA} (STAT5bCA) and Foxp3^{Cre-ERT2} (control) mice, nine mice for each experimental group, received a single dose (4 mg) of tamoxifen by oral gavage. Splenic CD4+ Foxp3(YFP/GFP)+GITRhiCD25hi Treg and CD4+ Foxp3(YFP/GFP)-CD62LhiCD44lo naïve T cells were double sorted using a BD FACSAria II cell sorter, and a total of 12 samples were generated. Spleen T cell subsets isolated from three individual mice in the same experimental group were pooled into one biological replicate; three biological replicates were subjected to RNA-seq analysis for each experimental group. Total RNA was extracted and used for poly(A) selection and Illumina TruSeq paired-end library preparation following manufacturer's protocols. Samples were sequenced on the Illumina HiSeq 2500 to an average depth of 27.5 million 50-bp read pairs per sample. All samples were processed at a same time and sequenced on the same lane to avoid batch effects.

Read alignment and processing followed the method previously described⁴⁵. Briefly, raw reads were trimmed using Trimmomatic v0.32 with standard settings to remove low-quality reads and adaptor contamination⁴⁶. The trimmed reads were then aligned to the mouse genome (Ensembl assembly GRCm³⁸) using TopHat2 v2.0.11 implementing Bowtie2 v2.2.2 with default settings. Read alignments were sorted with SAMtools v0.1.19 before being counted to genomic features using HTSeq v0.6.1p1. The overall read alignment rate across all samples was 74.5%. Differential gene expression was analyzed using DESeq2 1.6.3 in R version 3.1.0⁴⁷.

### Bioinformatic Analyses for RNA-Seq

The distribution of read counts across all genes was bimodal. The assumption that this corresponded to “expressed” and “non-expressed” genes was supported by examination of marker genes known to be expressed or not expressed in Treg and Tnaïve cells. The local minimum between the two peaks was chosen to be the threshold for expression. Using this threshold of ˜60 normalized reads, 10,589 out of 39,179 genes were called as present. Significantly up- (342 genes) and down-regulated (314) genes between STAT5bCA versus control Treg cells were defined as expressed genes with fold changes of at least 1.5× or 0.67×, respectively, and FDR-adjusted P-value≤0.05.

TCR-upregulated (i.e., TCR-dependent) genes were defined as genes downregulated (at least 0.57× fold change) in TCR-deficient compared to TCR-sufficient CD44hi Treg cells, while TCR-downregulated genes are upregulated (at least 1.75×, Padj≤0.001) in TCR-deficient CD44hi Treg cells (GSE61077)³⁴. Activation-upregulated genes are genes upregulated (2× fold change, Padj≤0.01) in Treg cells from Foxp3DTR mice recovering from punctual regulatory T cell depletion (GSE55753)³³.

Signaling Pathway Impact Analysis (SPIA) was performed using the R package of the same name⁴⁸. Significantly up- and downregulated genes, and their fold changes, were analyzed as one set for enrichment and perturbation of 90 Mus musculus KEGG pathways accessed on Oct. 5, 2015. The net pathway perturbation Z-score was calculated using the observed net perturbation accumulation, and the mean and SD of the null distribution of net perturbation accumulations. Global P-values were calculated using the normal inversion method with Bonferroni correction.

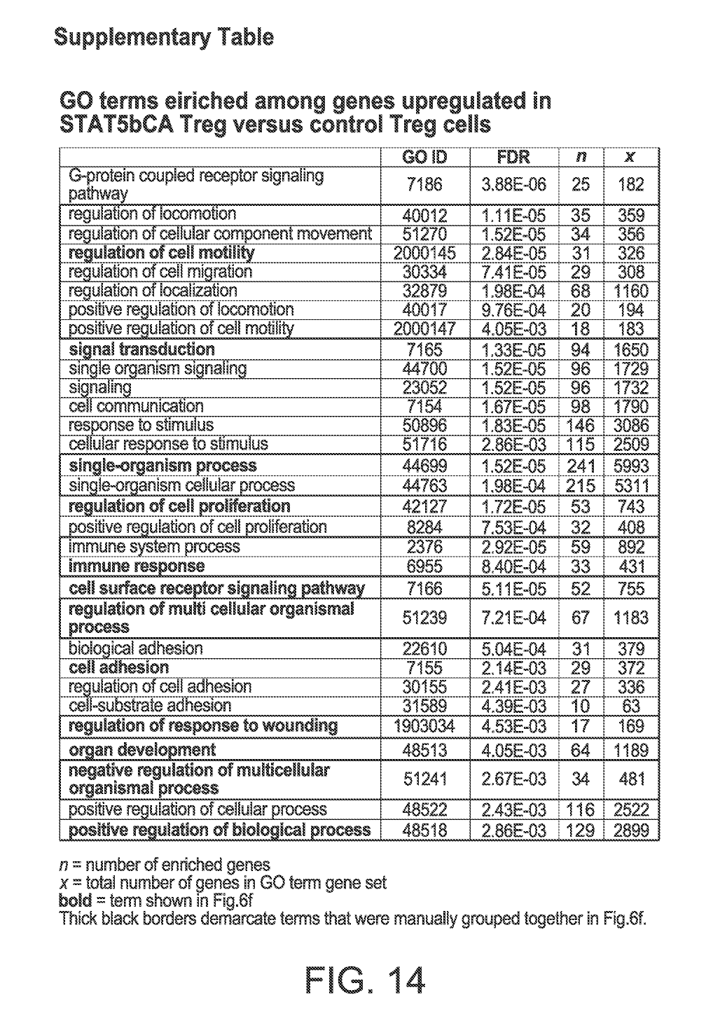

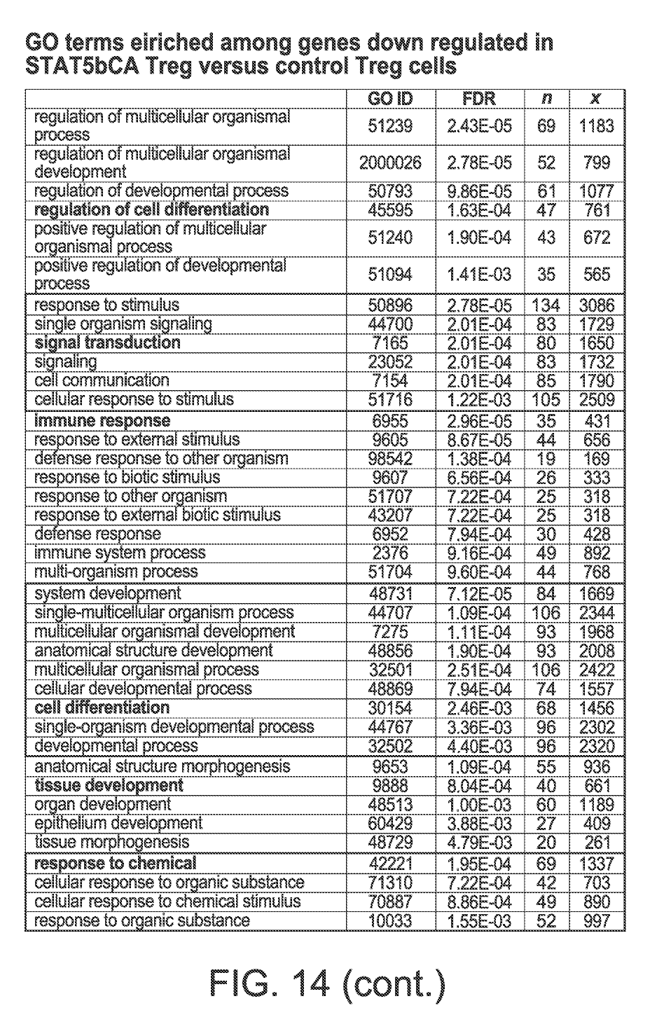

Biological process (BP) gene ontology (GO) term over-representation was calculated using BiNGO v3.0.3⁴⁹ in Cytoscape v3.2.1, employing the hypergeometric test and applying a significance cutoff of FDR-adjusted P-value≤0.05. The 10,589 expressed genes were entered as the reference set, and the GO ontology and annotation files used were downloaded on Oct. 25, 2015 (FIG. 14). The output from BiNGO was imported into EnrichmentMap v2.0.1⁵⁰ in Cytoscape to cluster redundant GO terms and visualize the results. An EnrichmentMap was generated using a Jaccard similarity coefficient cutoff of 0.2, a P-value cutoff of 0.001, an FDR-adjusted cutoff of 0.005, and excluding gene sets with fewer than 10 genes. The network was visualized using a prefuse force-directed layout with default settings and 500 iterations. Groups of similar GO terms were manually circled.

### Example 2: Role of IL-2 Receptor and STAT in Regulatory T-Cell Function

The present Example demonstrates that IL-2 capture is dispensable for control of CD4 T cells, but is important for limiting CD8 T cell activation, and that IL-2R dependent STAT5 activation plays an essential role in Treg suppressor function separable from TCR signaling.

Regulatory T (Treg) cells expressing the transcription factor Foxp3 restrain immune responses to self and foreign antigens¹⁻³. Treg cells express abundant amounts of the interleukin 2 receptor α-chain (IL-2Rα; CD25), but are unable to produce IL-2. IL-2 binds with low affinity to IL-2Rα or the common γ-chain (γc)/IL-2Rβ heterodimers, but receptor affinity increases ˜1,000 fold when these three subunits together with IL-2 form a complex⁴. IL-2 and STAT5, a key IL-2R downstream target, are indispensable for Foxp3 induction and differentiation of Treg cells in the thymus⁵⁻¹¹. IL-2Rβ and γc are shared with the IL-15 receptor, whose signaling can also contribute to the induction of Foxp3¹². IL-2, in cooperation with TGF-β, is also required for extrathymic Treg cell differentiation¹³.

While the role for IL-2R signaling in the induction of Foxp3 expression and Treg cell differentiation in the thymus has been well established by previous studies, the significance of IL-2R expression in mature Treg cells is not well understood. Although the deficiency in STAT5 abolishes Foxp3 expression, it can be rescued by increased amounts of the anti-apoptotic molecule Bcl2. This finding raised a possibility that a primary role for IL-2 is in the survival of differentiating Treg cells or their precursors¹³. It was also reported that Bim ablation can rescue Treg cells or their precursors from apoptosis associated with IL-2 or IL-2R deficiency and restore Treg cell numbers, but it did not prevent fatal autoimmunity¹⁵. However, a profound effect of a congenital deficiency in IL-2, Bcl2 and Bim on differentiation and selection of Treg self-reactive effector T cells confounds interpretation of this observation.

Antibody-mediated neutralization of IL-2 in thymectomized mice reduces Treg cell numbers and Foxp3 expression in Treg cells^{16,17}. Thus, IL-2 supports Treg cell lineage stability after differentiation^{18,19}. However, expression of a transgene encoding IL-2R□□ chain exclusively in thymocytes was reported to rescue the lethal autoimmune disease in Il2rb−/− mice, suggesting that IL-2R expression is dispensable in peripheral Treg cells7, 11. Thus, a role for IL-2R expression and signaling in peripheral Treg cells remains uncertain. Hypothetically, a role for IL-2R in peripheral Treg cells could be threefold: 1) guidance for Treg cells to sense their targets—activated self-reactive T cells, which serve as a source of IL-2; 2) Treg cell-mediated deprivation of IL-2 as a mechanism of suppression, and 3) cell-intrinsic IL-2 signaling in differentiated Treg cells to support their maintenance, proliferation, or function due to triggering of JAK-STAT5, PI3K-Akt, or Ras-ERK signaling pathways. Previous studies primarily focused on the induction or maintenance of Foxp3, while other aspects of IL-2R function have not been firmly established due to aforementioned limitations.

Despite their high reliance on IL-2 for the maintenance of Foxp3 expression, Treg cells are unable to produce IL-2. The reason for the inhibition of autologous activation of STAT5 in Treg cells, and potential biological significance of this IL-2-based Treg-Teff cell regulatory loop, also remain unknown. It has been suggested that repression of IL-2 is required to maintain the ‘unbound’ state of high affinity IL-2R on Treg cells, and unbound IL-2R serves a key role in Treg cell-mediated suppression by depriving Teff cells of IL-2²⁰⁻²⁴, however, whether this mechanism has a non-redundant role in suppression in vivo is unknown.

To address the role of IL-2R and downstream signaling pathways in differentiated Treg cells, we ablated of IL-2Rα, IL-2Rβ, and STAT5 in Foxp3-expressing cells. By simultaneously inducing expression of a constitutively active form of STAT5, we assessed the differential requirements for IL-2R expression and IL-2 signaling for Treg cell homeostasis vs. suppressor activity. We found that while continuous STAT5 signaling downstream of IL-2R maintained the expression of high affinity IL-2R, STAT5 activation completely abolished the requirement for IL-2R for the suppression of CD4+ T cells. However, capture of IL-2 by IL-2R expressed by Treg cells was indispensable for the suppression of CD8+ T cells. Our studies suggest that excessive STAT5 activation downstream of IL-2 signaling in CD8+ T cells confers resistance to Treg cell mediated suppression. STAT5 activation not only increased Foxp3 expression levels in Treg cells and promoted their expansion, but also potentiated their suppressor activity. Notably, the latter was increased even in the absence of TCR signaling. In addition to an essential role for IL-2 signaling in the induction and maintenance of Foxp3 expression and Treg cell numbers that has been shown in a large body of previous work, our studies demonstrated important and distinct roles for the IL-2R and STAT5 activation in the in vivo suppressor function of differentiated Treg cells.

### Results

### IL-2R is Indispensable for Treg Cell Function

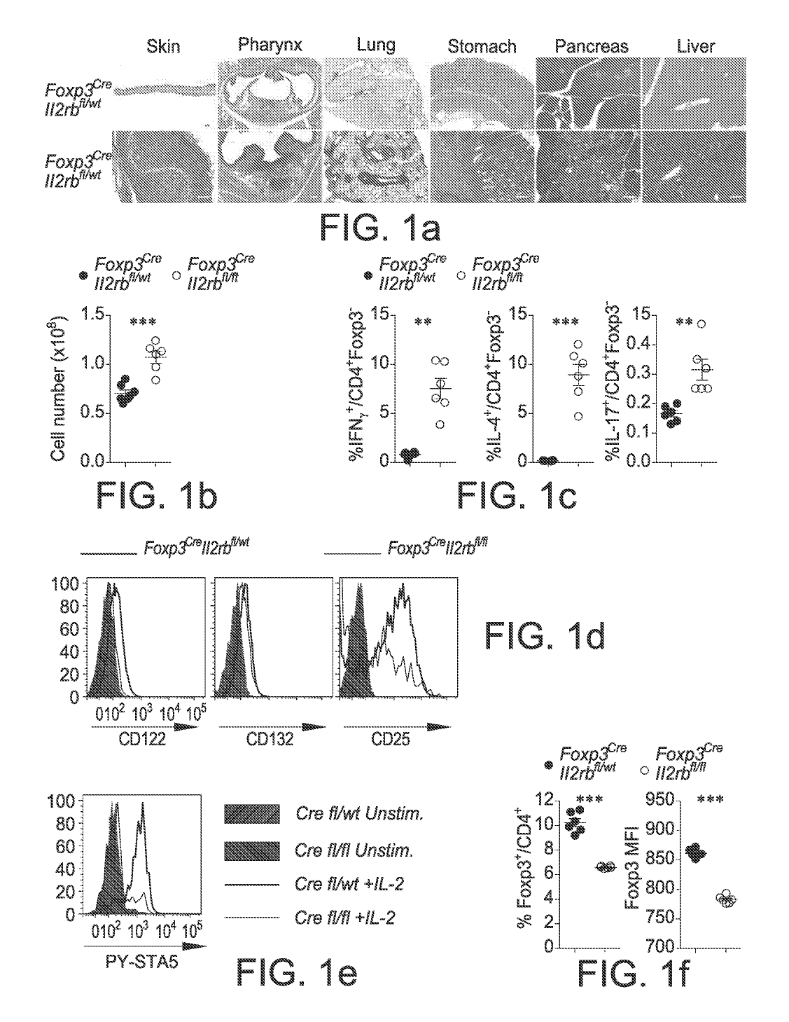

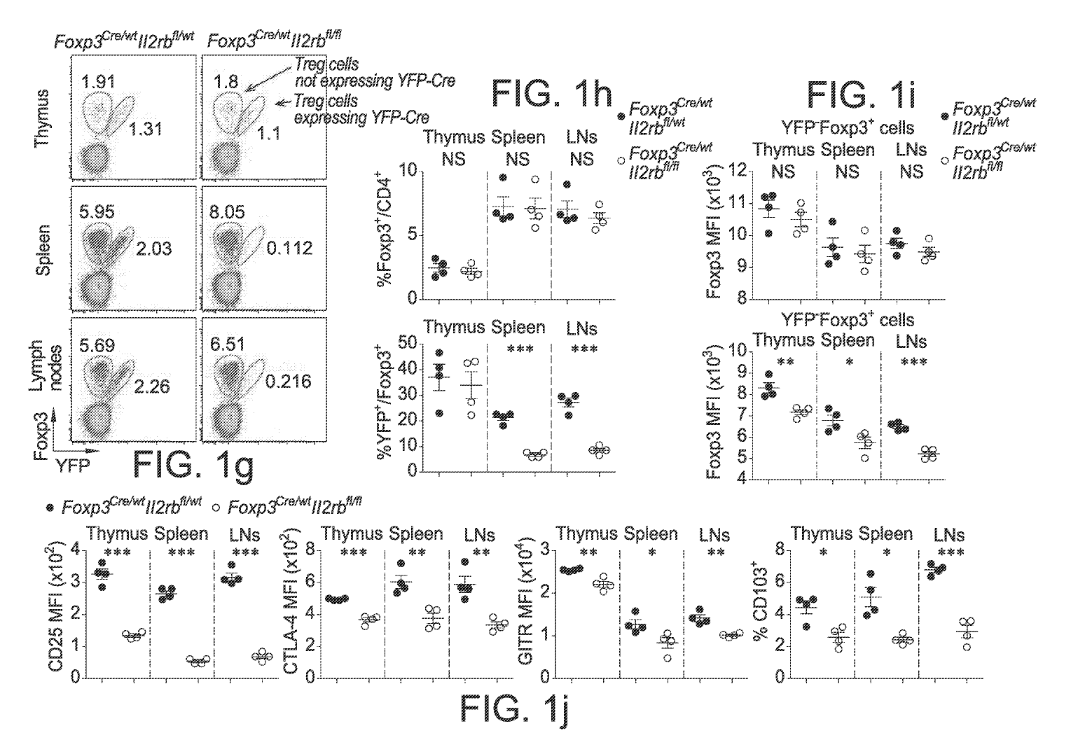

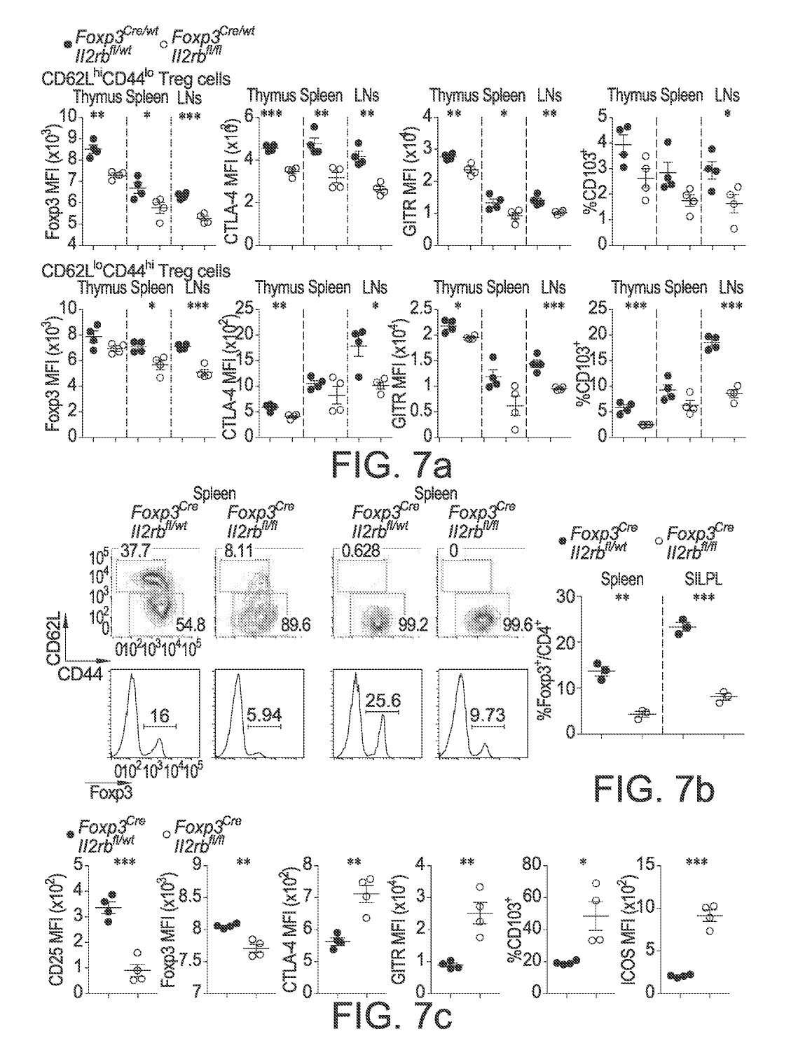

To establish a role for IL-2R in Treg cell function in vivo, we generated a conditional Il2rb allele and induced its ablation after Foxp3 was expressed using Cre recombinase driven by the endogenous Foxp3 locus (Foxp3^{Cre}). Il2rb^{fl/fl}Foxp3^{Cre} mice developed systemic fatal autoimmune inflammatory lesions and lymphoproliferation, albeit somewhat milder than that observed in Foxp3− mice (FIG. 1a-c). IL-2Rα expression was diminished in peripheral IL-2Rβ-deficient Treg cells (FIG. 1d), and tyrosine phosphorylation of STAT5 in response to IL-2 was lacking (FIG. 1e). The frequency of Foxp3+ cells among CD4+ T cells and the expression level of Foxp3 on a per-cell basis were both diminished (FIG. 1f). In healthy heterozygous Il2rb^{fl/fl}Foxp3^{Cre/WT} females, where both IL-2Rβ-sufficient (YFP+) and -deficient (YFP−) Treg cells co-exist due to random X-chromosome inactivation, IL-2Rβ-deficient Treg cells were underrepresented (FIG. 1g, h). It has been suggested that IL-2 is selectively required for the maintenance of CD62LhiCD44lo Treg cell subset, but is dispensable for CD62LloCD44hi Treg cells²⁵. However, we found both CD62LhiCD44lo and CD62LloCD44hi Treg cell subsets to be significantly reduced in the absence of IL-2Rβ in healthy heterozygous females. In these mice, IL-2Rβ-deficient Treg cells expressed reduced amounts of Foxp3 and Treg-cell “signature” molecules IL-2Rα chain (CD25), CTLA-4, GITR, and CD103 regardless of CD62L and CD44 expression (FIG. 1i, j and FIG. 7a). Although in diseased Il2rb^{fl/fl}Foxp3^{Cre} mice, a majority of Treg cells were CD62LloCD44hi, this was likely a consequence of severe inflammation because Treg cell frequencies were also markedly reduced at sites where CD62LloCD44hi cells were prevalent, i.e., the small and large intestines (FIG. 7b). Accordingly, many characteristic Treg cell markers, except for CD25 and Foxp3, were upregulated as the result of Treg cell activation in Il2rb^{fl/fl}Foxp3^{Cre} mice (FIG. 7c). These observations suggested that both CD62LhiCD44lo and CD62LloCD44hi Treg cell subsets, including those residing in the non-lymphoid tissues, are dependent on IL-2, though under inflammatory conditions the latter can be sustained to some extent by IL-2R-independent signals. Despite the upregulation of CTLA-4, GITR, ICOS, and CD103, the “activated” IL-2Rβ-deficient Treg cells from Il2rb^{fl/fl}Foxp3^{Cre} mice were still incapable of controlling inflammation in the diseased mice and were not suppressive when co-transferred with Teff cells into lymphopenic recipients (data not shown).

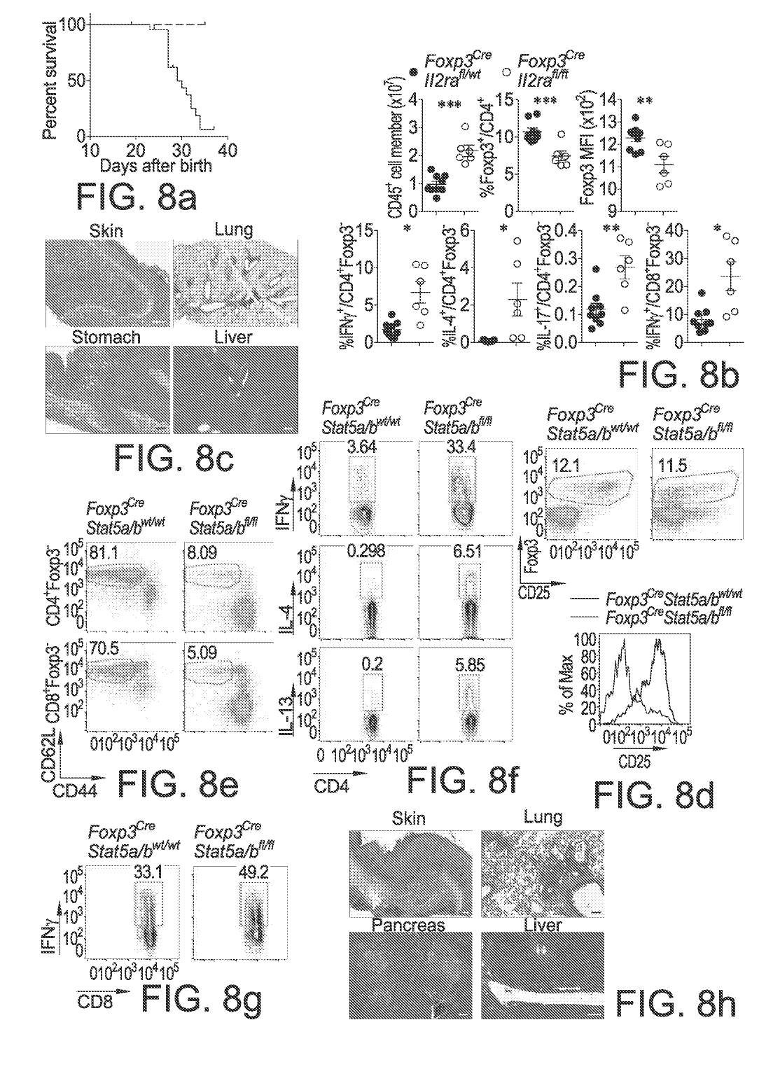

Our findings raised the question whether ablation of IL-2Rα, which, in addition to facilitating IL-2 signaling, enables its sequestration from Teff cells, would result in a similar Treg cell deficiency and disease compared to those in Foxp3^{Cre}Il2rb^{fl/fl} mice. Thus, we generated a conditional Il2ra allele and similarly induced its ablation in Treg cells. We found that Treg cell-specific IL-2Rα deficiency resulted in a disease with comparable early onset and severity to those observed upon IL-2Rβ ablation (FIG. 8a-c). Of note, germ-line deficiency of either Il2ra or Il2rb in mice on the same C57BL/6 background as our conditional knockout mice resulted in a considerably less aggressive disease with a delayed onset, likely due to a role for IL-2R signaling in Teff cells (data not shown). Our findings also indicate that IL-15 was unable to effectively compensate for the loss of IL-2 signaling in differentiated Treg cells because in Foxp3^{Cre}Il2ra^{fl/fl} mice, Treg cells lacked only IL-2 signaling, whereas in Foxp3^{Cre}Il2rb^{fl/fl} mice, they lacked both IL-2 and IL-15 signaling yet were similarly affected. This was in contrast to Treg cell differentiation in the thymus where IL-15 can contribute in part to Foxp3 induction12. Since IL-2R activates PI3K-Akt, MAPK, and JAK-STAT5 signaling pathways, we next sought to assess a role for STAT5 activation downstream of IL-2R signaling in Treg cells. We found that STAT5 ablation similarly impaired Treg cell function and Foxp3^{Cre}Stat5a/b^{fl/fl} mice were similarly affected by fatal autoimmunity as were mice harboring IL-2R deficient Treg cells (FIG. 8d-h).

### STAT5 Activation Rescues the Ability of IL-2R-Deficient Treg Cells to Suppress Lymphoproliferative Disease and CD4+ T Cell, but not CD8+ T Cell Activation

The above findings implied that STAT5 activation downstream of IL-2R is continuously required for Treg cell function. However, a marked decrease in IL-2R observed in STAT5-deficient Treg cells (FIG. 8d) made it impossible to separate a loss of STAT5 from impairment in all IL-2R functions, i.e., detection of IL-2, transduction of STAT5-dependent and -independent signals, and consumption and deprivation of IL-2, as a key contributor to the observed severe Treg cell dysfunction.

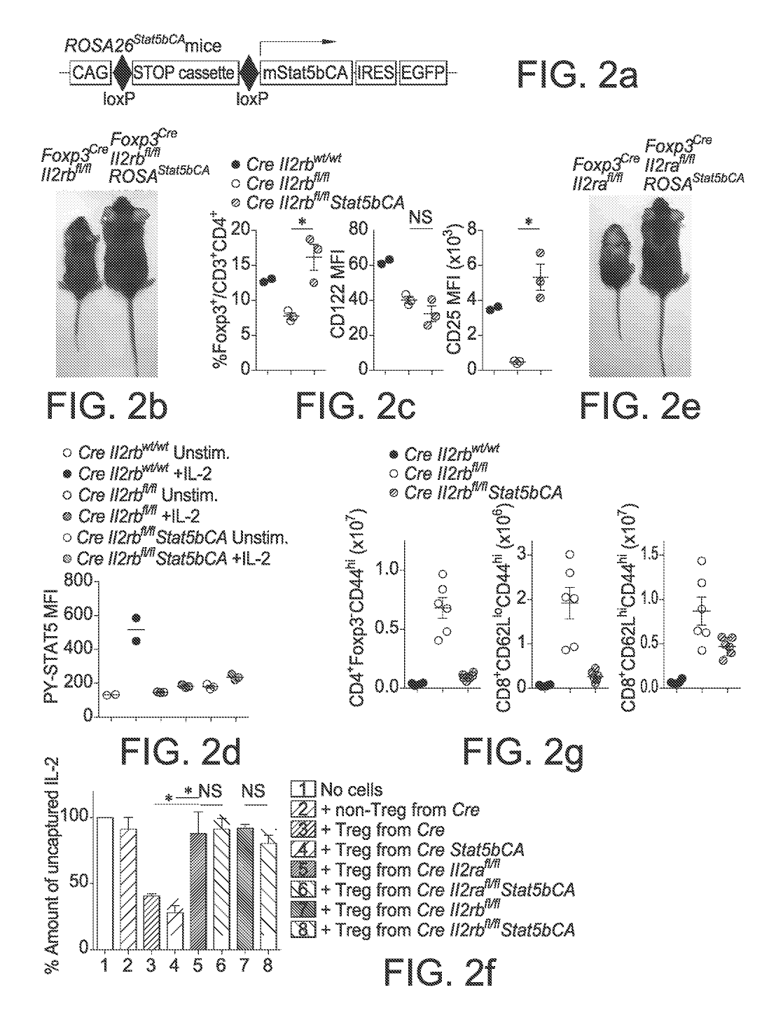

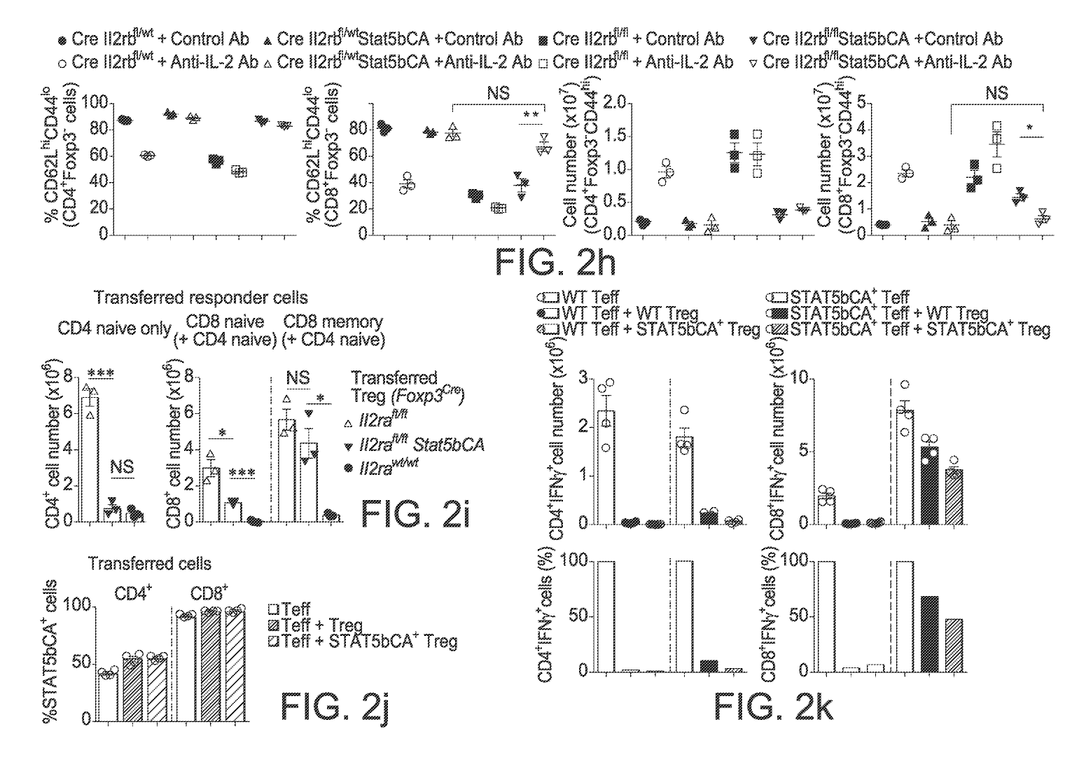

To address this major caveat and to understand a role for STAT5 vs. IL-2R, we asked whether expression of a gain-of-function form of STAT5b can rescue Treg cell function in the absence of IL-2R. A previous study using a transgene encoding a constitutively active form of STAT5b (STAT5bCA) driven by the proximal lck promoter in the absence of IL-2Rβ showed rescue of Treg cell differentiation in the thymus, but not lymphoproliferative syndrome⁹. However, the expression of this transgene early during thymopoiesis leads to leukemic lymphoproliferation, which complicated the interpretation of these findings. In addition, both the activity of the proximal lck promoter and the expression of the transgene diminish in peripheral T cells in these mice⁹. Therefore, we generated a gene-targeted mouse strain utilizing the ROSA26 “gene trap” locus26, where a CAG promoter driven STAT5bCA²⁷ is preceded by a loxP-flanked STOP cassette (FIG. 2a). In the resulting ROSA26^{Stat5bCA} mice, STAT5bCA is expressed only when the loxP sites undergo Cre mediated recombination. Introduction of the ROSA26^{Stat5bCA} allele into Foxp3^{Cre}Il2rb^{fl/fl} mice and the consequent expression of STAT5bCA in IL-2Rβ-deficient Treg cells rescued the systemic inflammation and early fatal disease (FIG. 2b). In these mice, Treg cell frequencies and numbers were comparable to or even surpassed their levels in wild-type (Foxp3^{Cre}) mice (FIG. 2c). Notably, the expression of IL-2Rα chain was increased despite the absence of IL-2Rβ chain (FIG. 2c), suggesting the expression of IL-2Rα on Treg cells is primarily controlled by STAT5-dependent, but not by STAT5-independent signaling. Importantly, these IL-2Rβ-deficient Treg cells with heightened IL-2Rα expression remained unresponsive to IL-2 (FIG. 2d).

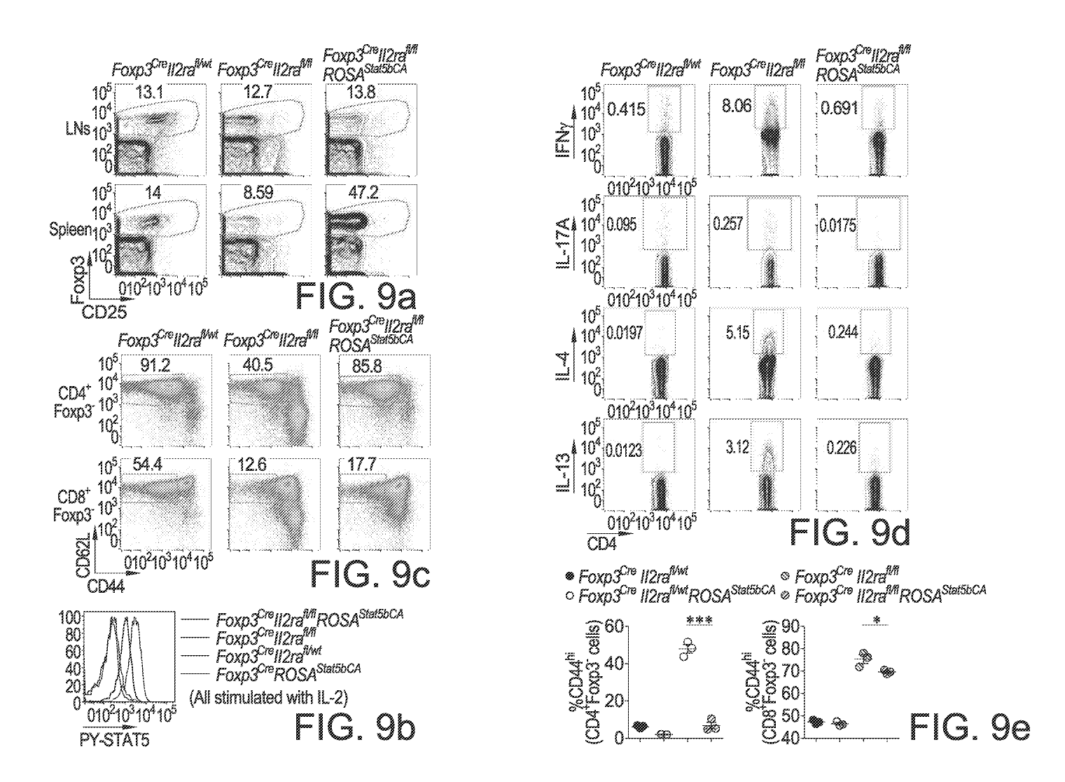

The observed restoration of the suppressor function of IL-2Rβ-deficient Treg cells and rescue of the early fatal disease upon STAT5bCA expression raised the possibility that the reintroduced high IL-2Rα levels were responsible for these effects. However, the expression of STAT5bCA similarly rescued the early fatal disease in Foxp3^{Cre}Il2ra^{fl/fl} mice (FIG. 2e and FIG. 9). Importantly, although the impaired capacity of Treg cells in both Foxp3^{Cre}Il2rb^{fl/fl} and Foxp3^{Cre}Il2ra^{fl/fl} mice to capture and consume IL-2 was not rescued upon STAT5bCA expression (FIG. 2f), CD4+ T cell reactivity was fully controlled in these mice (FIG. 2g and FIG. 9c-e). These results suggested that the ability to capture and compete for IL-2 is dispensable for Treg cell mediated suppression of CD4+ T cell responses. To the contrary, however expansion of CD8+ T cells, in particular, of activated CD62LhiCD44hi CD8+ T cells, was only marginally restrained in these mice (FIG. 2g and FIG. 9c, e)

Although the expansion of CD8+CD62LloCD44hi subset was relatively well, albeit not perfectly, controlled in neonatal mice (FIG. 2g and FIG. 9c), this subset also gradually started to expand in these mice as early as 2 to 3 wks after birth (data not shown). Although both Foxp3CreIl2rbfl/f ROSA26Stat5bCA and Foxp3CreIl2rafl/flROSA26Stat5bCA mice were rescued from premature death and showed significantly improved clinical status comparable to healthy controls, they gradually failed to thrive and started to succumb to disease accompanied by massively expanded activated CD62LhiCD44hi and CD62LloCD44hi CD8+ T cell subsets in LNs and tissues as early as 12 wk after birth (data not shown). These findings raised a possibility that IL-2 consumption by Treg cells, while dispensable for control of CD4+ T cells, is important for the restraint of CD8+ T cells.

IL-2 Consumption by Treg Cells is Essential for their Capacity to Suppress CD8+ T Cells In Vivo

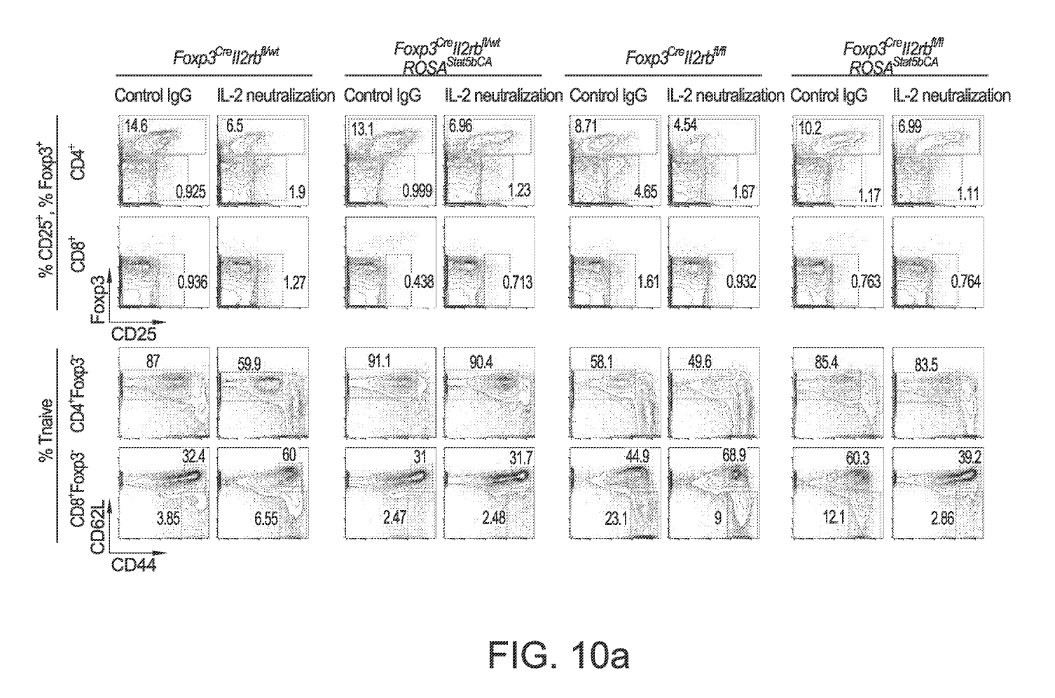

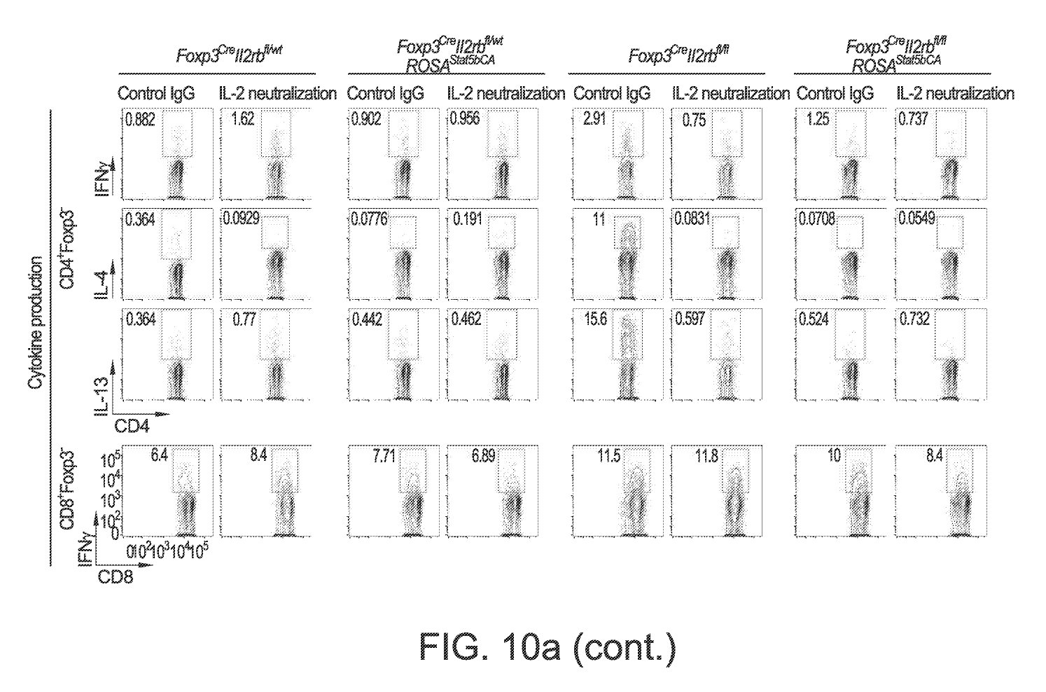

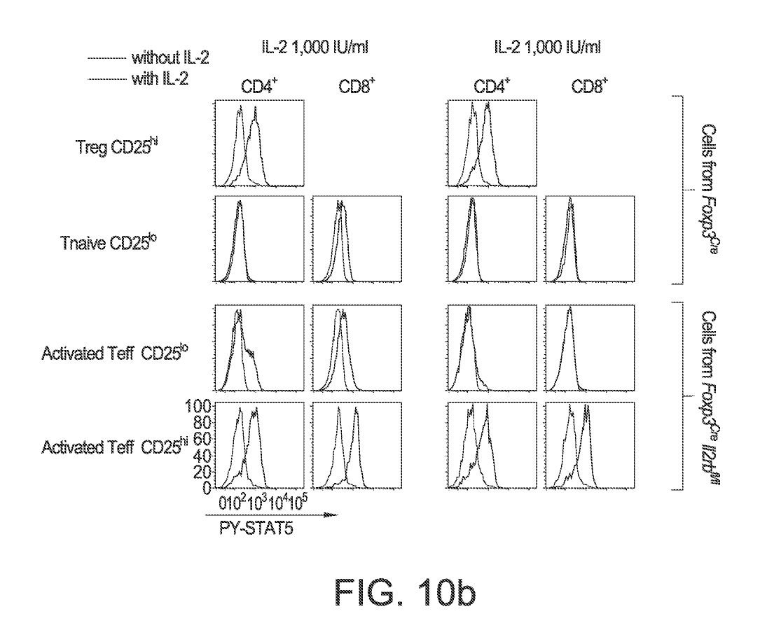

To test if the impairment in consumption of IL-2 by Treg cells can account for the expansion of CD8+ T cells in Foxp3^{Cre}Il2rb^{fl/f}lROSA26^{Stat5bCA} mice, we administered IL-2 neutralizing antibodies to these and control mice starting from 7 days of age (FIG. 2h and FIG. 10a). As IL-2 supports the differentiation of Treg cells in the thymus, IL-2 neutralization reduced the frequencies of Treg cells in all groups of mice and induced immunoactivation in control Foxp3^{Cre}Il2rb^{fl/wt} mice. In Foxp3^{Cre}Il2rb^{fl/fl} mice, which spontaneously develop disease, the production of Th2 cytokines IL-4 and IL-13 by CD4+ T cells was significantly reduced by IL-2 neutralization; however, the activation of CD4+ and CD8+ T cells was at best only marginally reduced or unaffected. In contrast, the activation and expansion of CD8+ T cells observed in Foxp3^{Cre}Il2rb^{fl/f}lROSA26^{Stat5bCA} mice were almost completely suppressed by the treatment.

The relative reduction in CD8+CD62LloCD44hi and more pronounced expansion of CD8+CD62LhiCD44hi T cell subset in Foxp3CreIl2rbfl/f ROSA26Stat5bCA and Foxp3CreIl2rafl/flROSA26Stat5bCA mice raised a possibility that a loss of IL-2-consumption by Treg cells might selectively impair their suppression for memory CD8+ T cell expansion, but not the recruitment of naïve CD8+ T cells into the effector cell pool. We tested this idea by adoptive transfer of CD4+ and CD8+ cell subsets into lymphopenic recipients (FIG. 2i). Consistent with the observation in Foxp3Cre mice, the impaired suppression of CD4+ T cell expansion and activation by IL-2R-deficient Treg cells was completely rescued by STAT5bCA; in contrast, their ability to suppress memory CD8+ T cells was not restored, whereas suppression of naïve CD8+ T cell expansion and expansion was only partially recovered. Thus, IL-2 consumption by Treg cells appears to have a non-redundant role in suppressing the expansion and activation of both naïve and memory CD8+ T cell subsets, although this mechanism appears to be particularly prominent in control of the latter subset.

Although the majority of activated CD8+ T cells in Foxp3^{Cre}Il2rb^{fl/fl} and Foxp3^{Cre}Il2rb^{fl/fl}ROSA26^{Stat5bCA} mice did not express detectable levels of IL-2Rα (FIG. 10a), these cells could activate STAT5 in response to IL-2, albeit to a lesser extent than that observed in cells expressing IL-2Rα (FIG. 10b). A proportion of activated CD4+ T cells with undetectable IL-2Rα expression also responded to IL-2, but the majority of them did not. CD8+ naïve T (Tnaïve) cells also responded to IL-2, while CD4+ Tnaïve cells did not. Thus, both naïve and activated CD8+ T cells appeared to be more sensitive to IL-2 than CD4+ T cells and IL-2 consumption by Treg cells may markedly affect their activation. A corollary to this notion was that STAT5 activation in CD8+, but not CD4+ T cells may render the former resistant to Treg cell mediated suppression. Thus, we tested the effect of STAT5 activation on the expansion of CD4+ and CD8+ T cells in the presence of Treg cells. For this purpose, we sorted CD4+ Foxp3− and CD8+ Foxp3− T cells from Foxp3^{Cre}ROSA26^{Stat5bCA} mice and induced the expression of STAT5bCA in these cells by treating them with a recombinant Cre protein containing a membrane permeable TAT peptide (TAT-Cre). We adoptively transferred the treated cells into lymphopenic recipients with or without Treg cells. Although TAT-Cre treatment initially induced STAT5bCA expression in approximately 30% of the treated CD4+ and CD8+ T cells, more than 95% of CD8+ T cells expressed STAT5bCA three weeks after the transfer; whereas STAT5bCA expressing CD4+ T cells expanded to 40-50% (FIG. 2j). Notably, STAT5bCA+CD8+ T cells robustly expanded in the presence of either wild-type (Foxp3^{Cre}) or STAT5bCA+ Treg cells (FIG. 2j, k). Although some degree of suppression of STAT5bCA+CD8+ T cells by Treg cells was still observed, it was very mild compared to the suppression of STAT5bCA-CD8+ T cells (FIG. 2k) In contrast, proliferation of and cytokine production by activated CD4+ T cells, regardless of the expression of STAT5bCA, were well controlled by Treg cells. These observations suggest that STAT5 activation in CD8+, but not in CD4+ T cells prompts robust expansion of cells and confers pronounced resistance to Treg cell mediated suppression. Consistent with these findings, gain-of-function experiments where IL-2 was provided in the form of IL-2/anti-IL-2 immune complexes showed expansion of CD8+T and CD4+ Treg, but not of CD4+ T cells28. Thus, while the ability to capture and compete for IL-2 is dispensable for Treg cell mediated suppression of CD4+ T cell responses, this mode of suppression appears essential for control of CD8+ T cells, which respond to excessive IL-2 more robustly than CD4+ T cells.

### Autonomous Activation of STAT5 in Treg Cells Boosts Immunosuppression

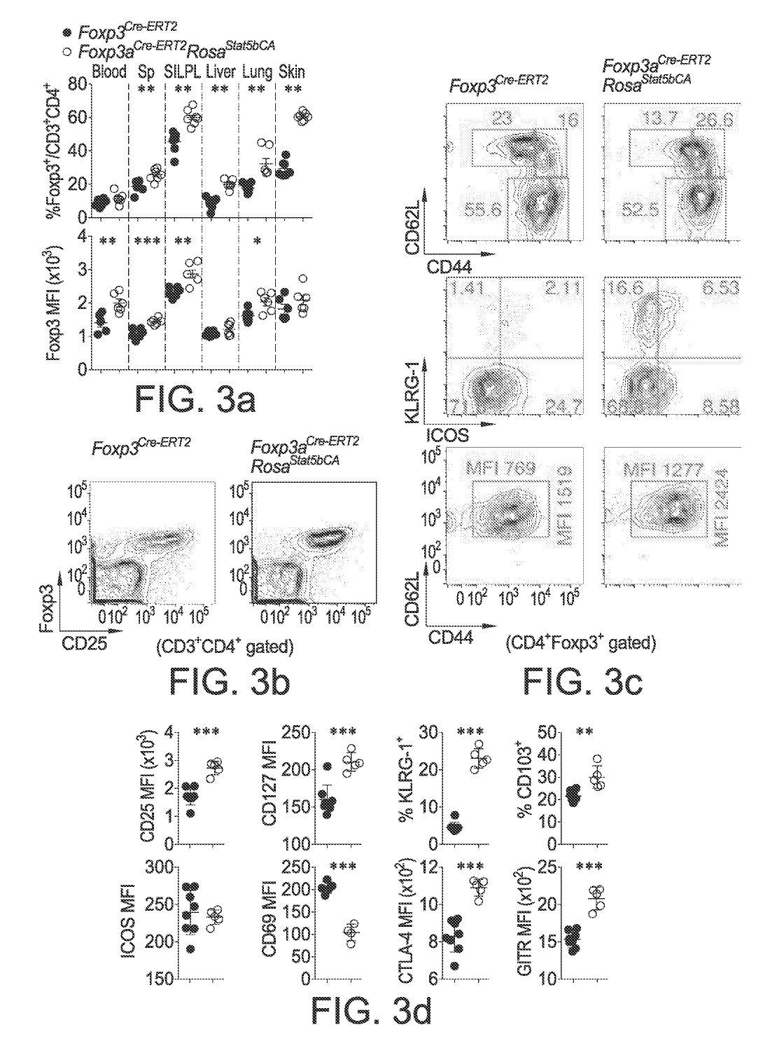

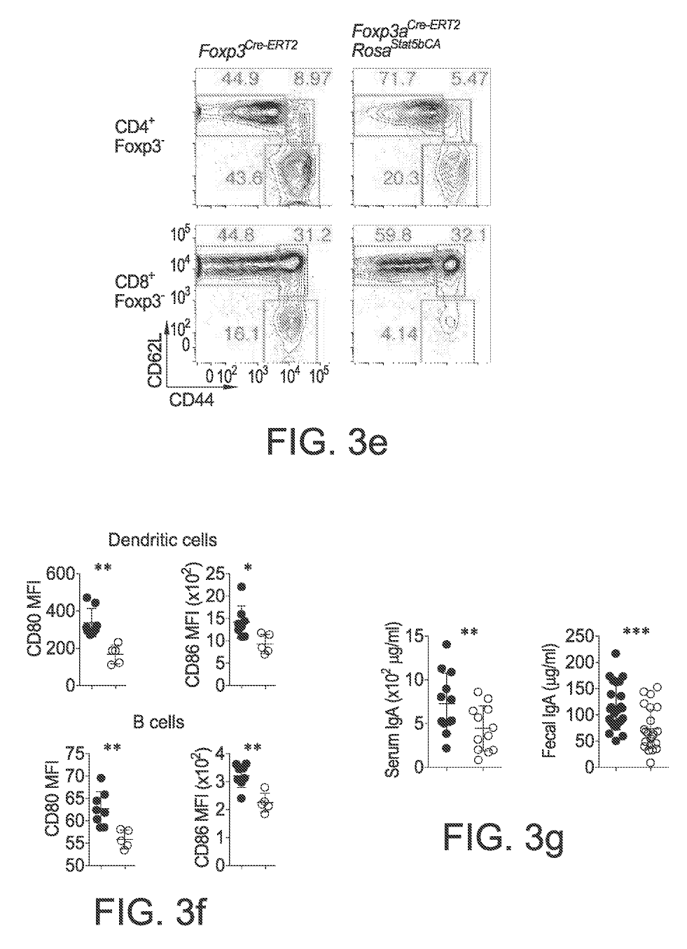

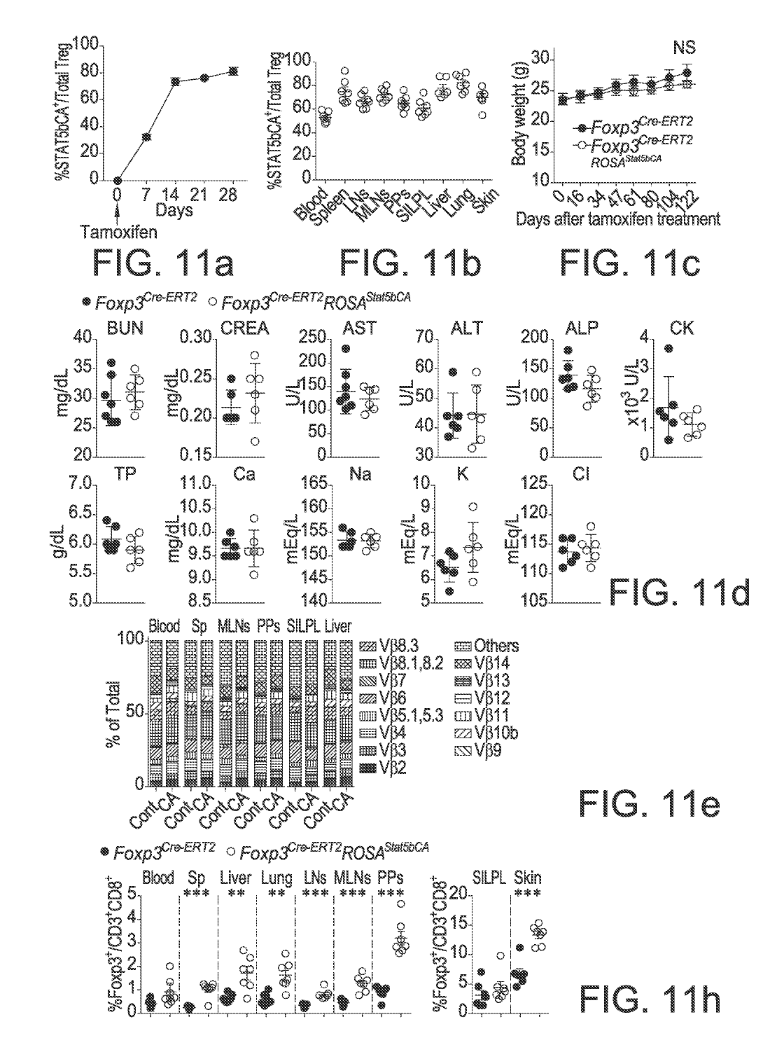

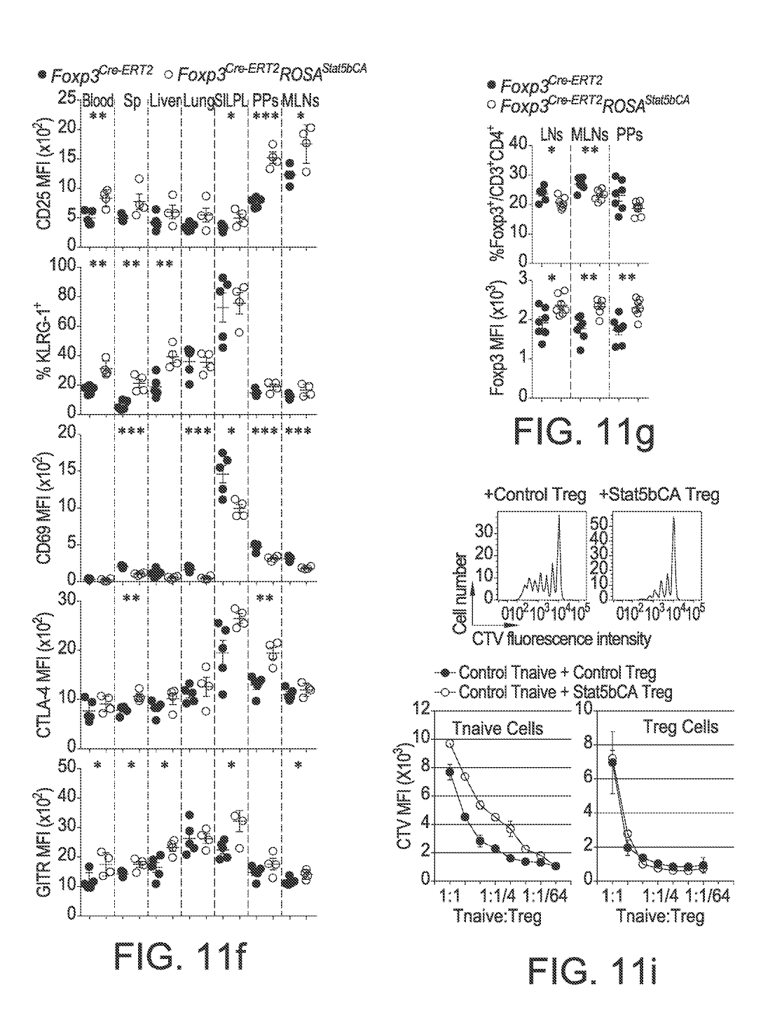

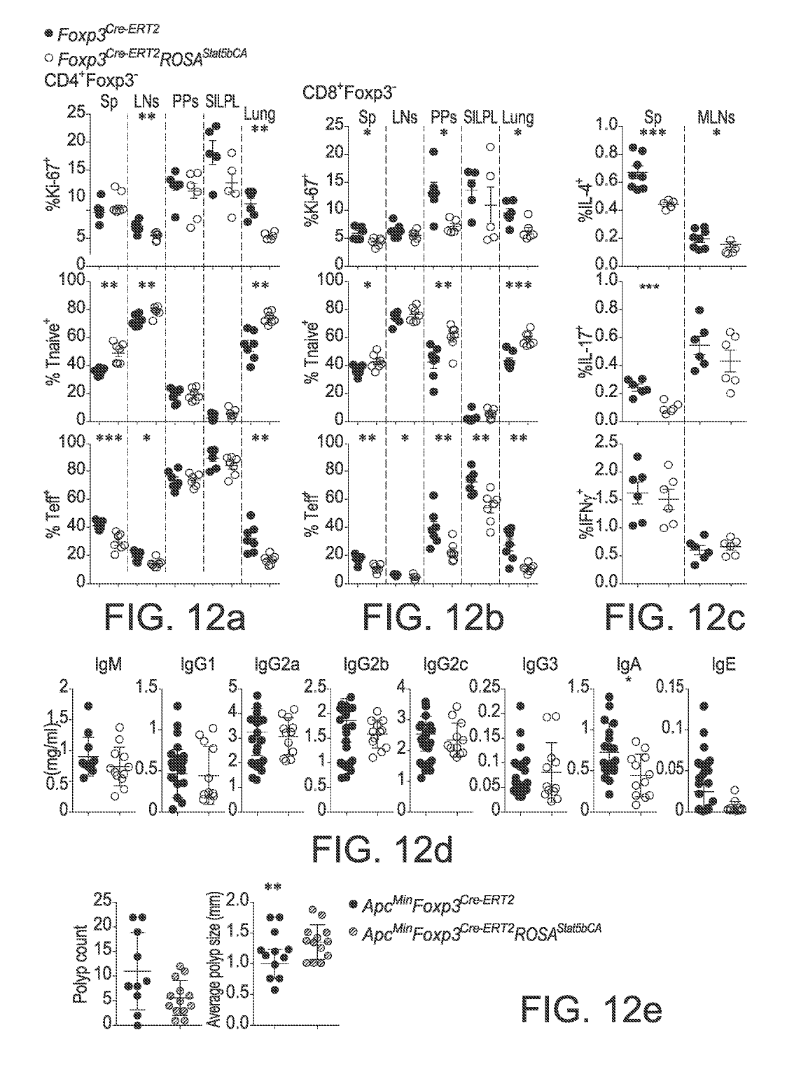

The lack of detectable STAT5 activation in response to IL-2 and of STAT5bCA-driven expansion of IL-2R-sufficient Treg cells that escaped from Cre-mediated recombination (counter-selection) in both Foxp3^{Cre}Il2rb^{fl/fl}ROSA26^{Stat5bCA} and Foxp3^{Cre}Il2ra^{fl/fl}ROSA26^{Stat5bCA} mice indicated that the expression of a constitutively active form of STAT5 relieved Treg cells from their dependence on IL-2 signaling. This finding offered a unique opportunity to explore the biological significance of the aforementioned IL-2-dependent Treg-Teff cell regulatory network by uncoupling Treg cell function from IL-2 production by Teff cells. To address this question, we generated ROSA26^{Stat5bCA}Foxp3^{Cre-ERT2} mice, which enabled tamoxifen-inducible expression of STAT5bCA in differentiated Treg cells¹⁶. Induction of STAT5bCA expression in ˜20-30% of Treg cells upon a single tamoxifen administration was followed by their rapid increase in numbers at the expense of Treg cells with a non-recombined ROSA26^{Stat5bCA} allele (FIG. 11a, b). The experimental Foxp3^{Cre-ERT2}ROSA26^{Stat5bCA} mice remained healthy (FIG. 11c, d). In these mice, the expanded STAT5bCA+ Treg cell population exhibited increased amounts of Foxp3, CD25, CTLA4, and GITR and an increased proportion of CD62LhiCD44hi vs. CD62LhiCD44lo cells, indicative of a STAT5bCA impressed biasing of the Treg cell population towards an activated or “memory” cell state (FIG. 3a-d, FIG. 11f). Consistent with the latter possibility, the expression levels of IL-7R, KLRG1, and CD103 were increased (FIG. 3d). It is noteworthy that these cells exhibited a highly diverse TCR Vβ usage similar to that in control mice (FIG. 11e). CD8+ Foxp3+ cells were also increased upon induction of STAT5bCA (FIG. 11h). The “autonomous” Treg cells, expressing active STAT5, effectively suppressed the basal state of activation and proliferative activity of CD4+ and CD8+ T cell subsets as evidenced by the decreased numbers of Ki-67+ cells and CD62LloCD44hi Teff cells and a markedly increased CD62LhiCD44lo Tnaïve cell pool (FIG. 3e and FIG. 12a,b). Notably, in lymph nodes (LNs) and Peyer's patches (PPs), Treg cells were not numerically increased despite the predominance of STAT5bCA+ Treg cells (FIG. 11b, g); however, Teff cell responses in these tissues were also diminished (FIG. 12a, b), suggesting the increased suppressor function conferred by a constitutively active form of STAT5. In vitro suppression assay also revealed heightened suppressor activity of STAT5bCA+ Treg cells (FIG. 11i). Correspondingly, CD4+ T cell production of pro-inflammatory cytokines, most prominently IL-4, and expression of CD80 and CD86 by B cells and dendritic cells (DCs) were reduced (FIG. 12c and FIG. 3f). Previously, Treg cells were proposed to promote systemic Th17 type responses and IgA class switching in the gut ^{29,30}. However, we found that serum and fecal IgA as well as Th17 responses in secondary lymphoid organs were reduced, rather than increased in the presence of STAT5bCA+ Treg cells (FIG. 3g and FIG. 12c). Serum IgM and IgE also showed a tendency towards a decrease, but this was not statistically significant (FIG. 12d). These results were in agreement with an increase in Th17 responses and in both Th2- and Th1-type Ig class switch observed upon acute Treg cell ablation³¹. Since altered intestinal immune responses have been implicated in promoting colonic carcinogenesis, we explored an effect of a gain in Treg cell suppressor function afforded by activated STAT5 in an Apc^{Min} model of colorectal cancer. Mice harboring the Apc^{Min} mutation develop multiple adenomatous polyps in the small intestine³². Apc^{Min}Foxp3^{Cre-ERT2}ROSA26^{Stat5bCA} mice developed a comparable or fewer numbers of polyps, but the average polyp size was increased (FIG. 12e). These results were consistent with the idea that suppression of inflammation by Treg cells in tumor microenvironments promotes the growth of tumors once tumors or pre-cancerous lesions are already formed. However, the early stages of colonic carcinogenesis appeared not to be promoted but were potentially suppressed by Treg cells with augmented suppressor activity.

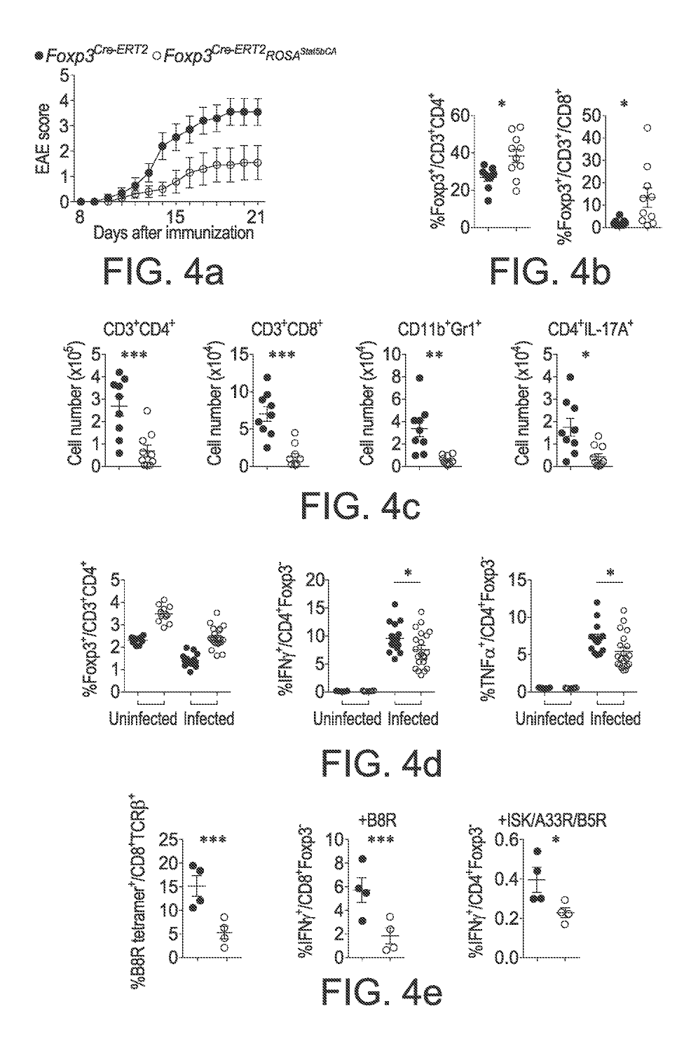

In addition to restraining the basal immune reactivity in physiological settings and modulating colon carcinoma development, “autonomous” Treg cells afforded superior protection against autoantigen-induced autoimmunity. We found that Foxp3^{Cre-ERT2}ROSA26^{Stat5bCA} mice were highly resistant to experimental autoimmune encephalomyelitis (EAE) (FIG. 4a-c). The frequencies of CD4+ Foxp3+ cells were significantly increased in the brain and spinal cord of these mice (FIG. 4b), and infiltration of inflammatory cells, including neutrophils and IL-17-producing CD4+Th17 cells into these organs, was significantly reduced (FIG. 4c). Pathogen-specific responses were also diminished in Foxp3^{Cre-ERT2}ROSA26^{Stat5bCA} mice. While Listeria-specific Th1 responses were only modestly suppressed (FIG. 4d), vaccinia virus-specific CD8+ T cell responses were markedly decreased in the presence of STAT5bCA+ Treg cells (FIG. 4e). Our observation of diminished responses to infectious agents and modulation of cancer progression may provide teleologic rationale as to why Treg cells are lacking in IL-2 production and autonomous activation of STAT5, and instead are reliant on activated T cells as a source of IL-2.

### A TCR-Independent Role of STAT5 Signaling in Treg Cell Gene Expression and Suppressor Function.

Next, we sought to address the question of how sustained STAT5 signaling may potentiate Treg cells' ability for suppression. In genetic loss- and gain-of-function studies, STAT5 activity in Treg cells correlated with their proliferative capacity and expression levels of IL-2Rα and Foxp3. However, the aforementioned results of in vitro suppression assay, as well as the reduction in immune activation in LNs and PPs of Foxp3Cre-ERT2ROSA26^{Stat5bCA} mice, where fewer Treg cells were found than in control mice, suggested that the enhanced immunosuppression observed in Foxp3^{Cre-ERT2}ROSA26^{Stat5bCA} mice was not simply due to a numerical increase of Treg cells, but that their suppressor activity on a per cell basis was also augmented. It is also unlikely that mild upregulation of Foxp3 in the presence of STAT5bCA can account for the increased suppressor activity of Treg cells as we found that genome-wide Foxp3 binding does not change upon activation of Treg cells, which lead to an increase in Foxp3 expression more pronounced than the one caused by STAT5bCA³³. The increase in Foxp3 expression levels in STAT5bCA+ Treg cells compared to control was particularly pronounced in the CD2510 Treg cell subset (FIG. 3b), consistent with the observation that STAT5bCA+ Treg cells were relieved from their dependence on IL-2. Nevertheless, STAT5bCA+ Treg cells exhibited a more potent suppressor function than CD25hiFoxp3hi Treg cells from control mice when co-transferred with effector T cells into lymphopenic recipients than CD25hiFoxp3hi Treg cells from control mice despite comparably high expression of Foxp3 (data not shown). Thus, the increased suppressor activity of STAT5bCA+ Treg cells cannot be ascribed to the increased levels of Foxp3.

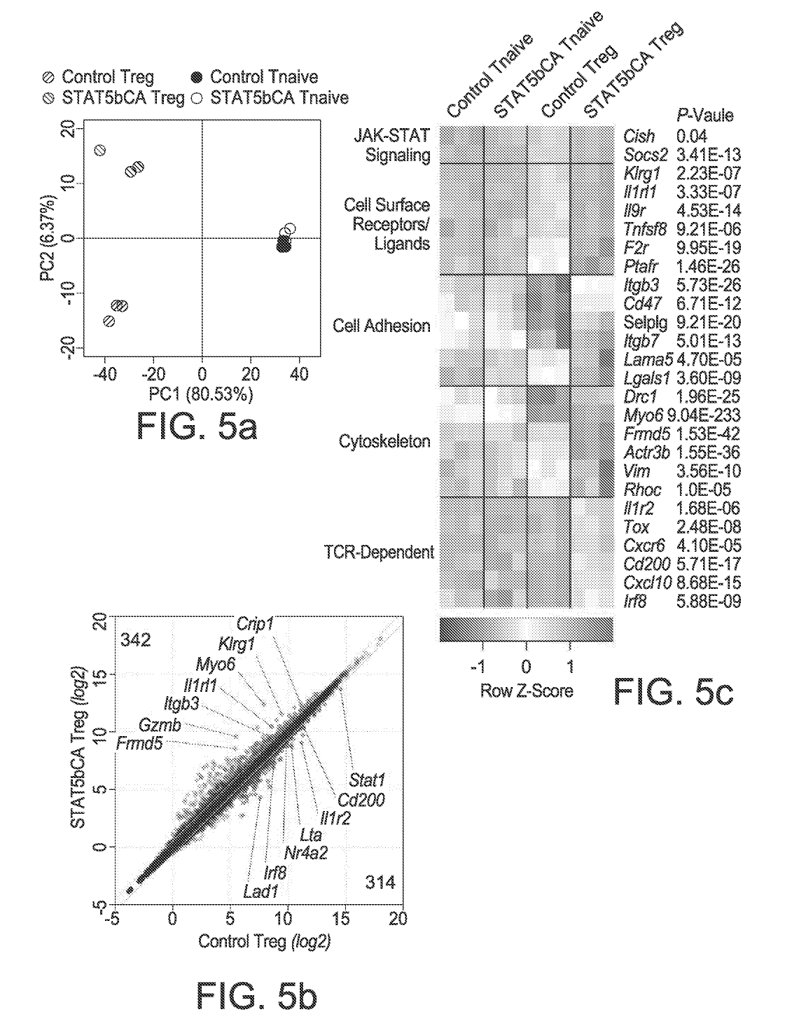

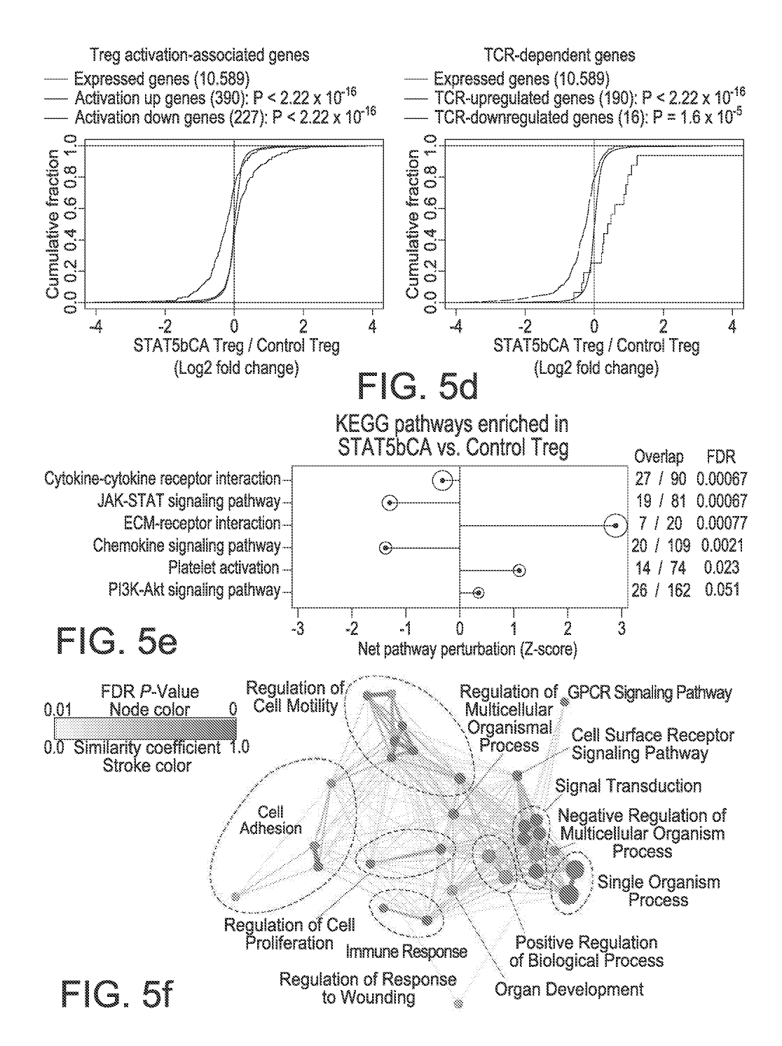

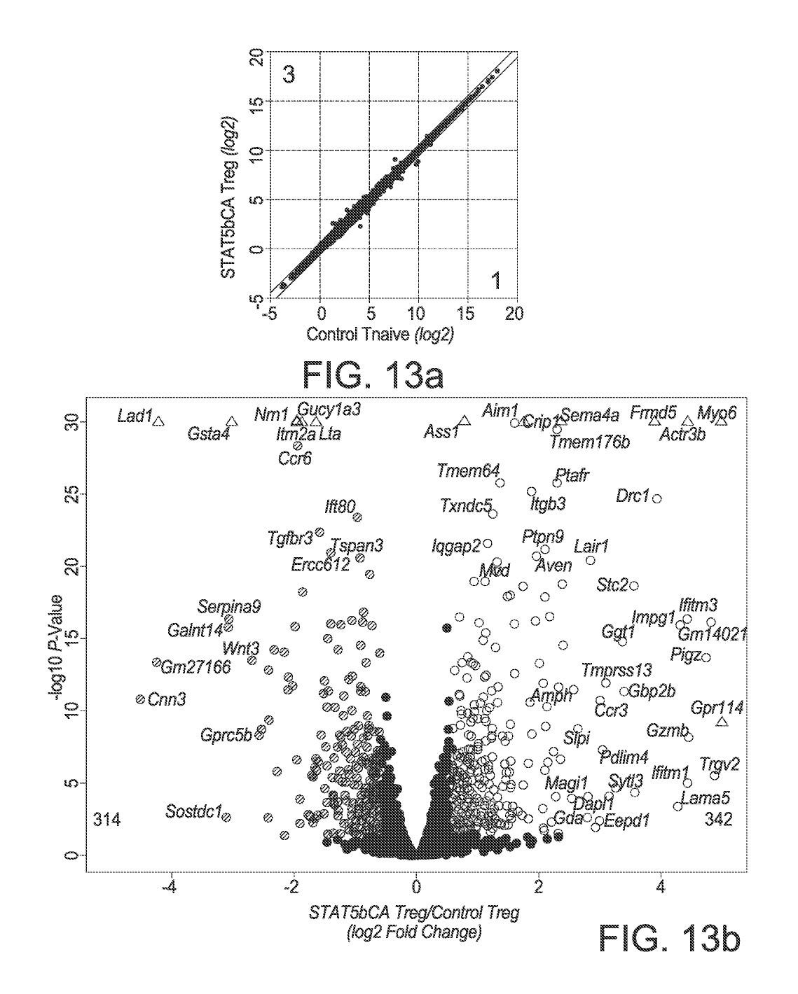

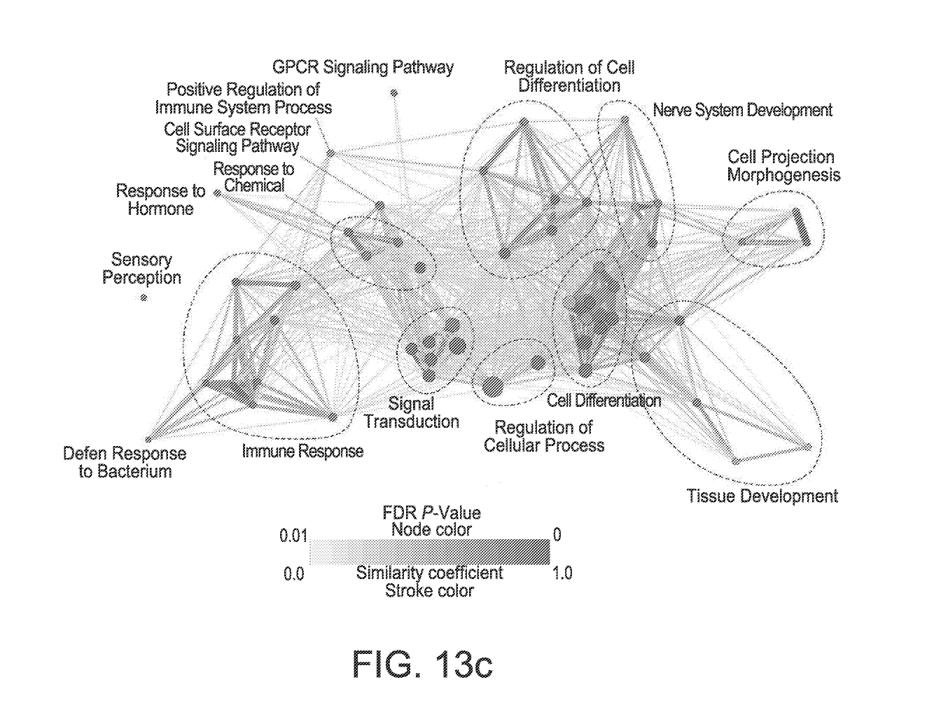

To gain insight into the potential mechanisms underlying the heightened suppressor function conferred by sustained STAT5 activation, we sorted mature Treg cells from Foxp3^{Cre-ERT2} and Foxp3^{Cre-ERT2}ROSA26^{Stat5bCA} mice that expressed comparable levels of Foxp3 and analyzed gene expression in these cells using RNA-seq. While the gene expression profiles of CD4+ Tnaïve cells from both groups of mice were nearly identical, Treg cell gene expression was markedly affected by the active form of STAT5 (FIG. 5 and FIG. 13a). Among all expressed genes (˜11,000) in either Treg or CD4+ Tnaïve cell populations analyzed, 342 genes were upregulated and 314 genes were downregulated in STAT5bCA+ Treg cells compared to control cells (FIG. 5b and FIG. 13b). The gene set upregulated in STAT5bCA+ Treg cells encoded various cell surface molecules and receptors involved in cell adhesion, migration, and cytoskeletal reorganization (FIG. 5c). Several genes that were upregulated or downregulated in control Treg cells compared to Tnaïve cells showed opposite trends in STAT5bCA+ Treg cells, suggesting that STAT5bCA does not simply reinforce the Treg cell signature. Our recent study showed that exposure of Treg cells to inflammation induced upon transient Treg cell depletion leads to a marked change in their gene expression and a potent increase in their suppressor function³³. Consistent with the heightened suppressor function of STAT5bCA+ Treg cells, we found that the gene expression changes in these cells conferred by a constitutively active form of STAT5 correlated with those found in highly activated Treg cells in inflammatory settings (FIG. 5d). Previously, we found that TCR signaling is required for the ability of Treg cells to exert their suppressor function^{34, 35}. Thus, it was possible that TCR and STAT5 dependent signaling pathways in Treg cells are acting upon a largely overlapping set of genes whose expression they jointly regulate to potentiate Treg cell suppressor activity. However, our analysis revealed that the gene set affected by the active form of STAT5 was distinct from that expressed in Treg cells in a TCR-dependent manner (FIG. 5d). Thus, both TCR and STAT5 signaling pathways play an indispensable role in Treg cell suppressor activity in vivo by controlling largely distinct sets of genes and likely distinct aspects of Treg cell suppressor activity.

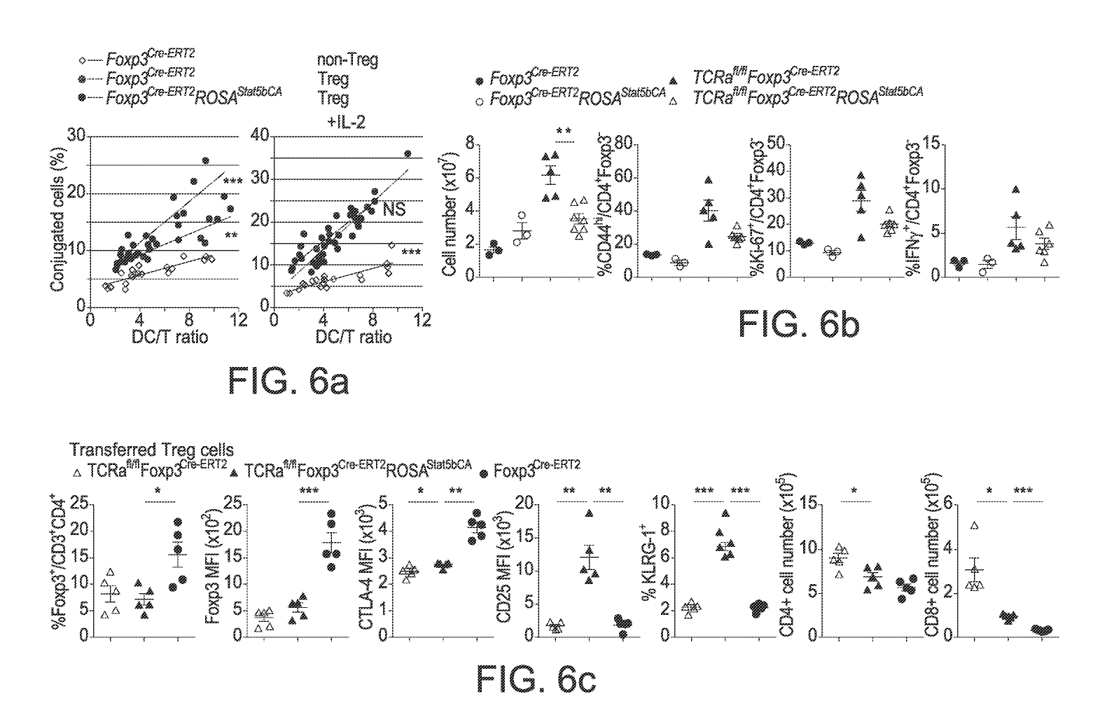

To better understand aspects of Treg cell function potentiated by STAT5 activation, we performed signaling pathway and molecular function enrichment analyses, which revealed overrepresentation of gene sets involved in cell-cell and extracellular matrix interactions, cell adhesion, and cellular locomotion among genes differentially expressed in STAT5bCA+ Treg cells (FIG. 5e, f). This result suggested that in Treg cells, STAT5 activation might potentiate their interactions with the target cells. Since intravital imaging of Treg cells in vivo had previously revealed their stable interactions with DCs36, we assessed the potential effect of constitutively active STAT5 expression in Treg cells on their ability to form conjugates with DCs in vitro. In agreement with the gene set enrichment analysis, we found that expression in Treg cells promotes conjugate formation between Treg and DCs (FIG. 6a). Heightened interactions of STAT5bCA+ Treg cells with DCs in vitro were consistent with the decreased expression of co-stimulatory molecules by DCs observed in tamoxifen-treated Foxp3^{Cre-ERT2}ROSA26^{Stat5bCA} mice.

These findings raised a question whether STAT5 activation can potentiate the suppressor function of Treg cells in a TCR-independent manner. To test this notion, we analyzed Foxp3^{Cre-ERT2}ROSA26^{Stat5bCA} mice expressing a conditional Tcra allele. As we reported previously, tamoxifen-inducible Cre-mediated TCR ablation resulted in immune activation resulting from impaired suppressor function³⁴. Interestingly, the marked increase in T cell activation and pro-inflammatory cytokine production was mitigated in part upon expression of the active form of STAT5 in tamoxifen-treated Foxp3^{Cre-ERT2T} Tcra^{fl/fl}ROSA26^{Stat5bCA} mice (FIG. 6b). This partial recovery of Treg cell suppressor function by the active form of STAT5 in TCR-ablated Treg cells was also confirmed in experiments where FACS-purified TCR-deficient STAT5bCA+ Treg cells and effector T cells were adoptively transferred into lymphopenic recipients (FIG. 6c). Although the rescue was incomplete, these results suggested that enhanced STAT5 signaling could potentiate Treg cell suppressor activity in the absence of contemporaneous TCR-dependent signals. Indeed, some features of Treg cells that had been observed in TCR-sufficient STAT5bCA+ Treg cells were still present in TCR-ablated STAT5bCA+ Treg cells (FIG. 6c). It should be noted, however, that STAT5bCA expression failed to rescue suppressor function in Foxp3^{Cre}Tcra^{fl/fl}ROSA26^{Stat5bCA} mice where TCR deletion occurred immediately after the induction of Foxp3. We have previously shown that TCR signal is required for Treg cells to acquire activated, antigen-experienced phenotype and suppressor function³⁴. Thus, our results suggest that activation of STAT5 potentiates TCR-independent suppressor function in mature Treg cells that have already undergone TCR-dependent maturation. This observation is reminiscent of the sequential requirement for these two signals, TCR and IL-2R, in the differentiation of Treg cells in the thymus where STAT5 signal promotes differentiation of Treg precursors that have experienced permissive TCR signaling³⁷. Discussion

The discovery of high cell-surface amounts of IL-2Rα as a distinguishing feature of a CD4+ T cell subset with suppressor function set the stage for extensive investigation of the role of IL-2 and IL-2R signaling in Treg cell biology over the last two decades. Previous analysis of mice with germ-line deficiency in IL-2 and IL-2R subunits demonstrated that IL-2 is a key cytokine required for the induction of Foxp3 and the differentiation of Treg cells in the thymus⁵⁻¹¹. Furthermore, antibody-mediated IL-2 neutralization and provision of IL-2 in the form of immune complexes with a stabilizing IL-2 antibody, as well as genetic dissection of regulatory elements within the Foxp3 locus, revealed an important role for IL-2 in the maintenance in mature Treg cells and in stabilization of Foxp3 expression during their extrathymic differentiation^{16, 28, 37}. These findings raised a question of whether IL-2R signaling can also directly promote Treg cell suppressor capacity and, therefore, serve as a critical nexus linking differentiation and maintenance of Treg cells with their suppressor function. An early in vitro study proposed a role for IL-2 signaling based on indirect evidence²¹. In addition, IL-2 consumption by Treg cells was suggested to play an essential role in Treg cell suppressor function by causing death of activated CD4+ T cells due to IL-2 deprivation²⁰⁻²⁴. On the other hand, several other reports suggested that IL-2R is dispensable for the ability of Treg cells to suppress effector T cell proliferation^{8, 39}. Furthermore, the rescue of the disease in Il2ra−/− and Il2rb−/− mice observed upon adoptive transfer of wild-type Treg cells suggested the existence of major mechanisms of Treg cell-mediated suppression independent of IL-2-deprivation^{6,7}. However, the latter studies left open a major question as to whether IL-2 consumption by Treg cells is essential for suppression of IL-2R-sufficient Teff cells since IL-2 is likely a major driver of autoimmune disease in the absence of functional Treg cells.

A major limiting factor in efforts to experimentally assess a role for IL-2R signaling in, and IL-2 consumption by Treg cells in their function in vivo has been the lack of adequate genetic tools. The use of mice with a germ-line IL-2R deficiency in these studies has been confounded by the impairment in the Foxp3 induction, early differentiation of hematopoietic cell lineages including T and B cells, survival of Treg precursors prior to Foxp3 expression, and potential perturbation of the Treg TCR repertoire. We addressed these issues through generation of conditional Il2ra and Il2rb alleles and their ablation in Treg cells in combination with the induced expression of a constitutively active form of STAT5. These new genetic tools enabled us to unequivocally demonstrate that IL-2R signaling has a cell intrinsic, non-redundant role not only in the maintenance of mature Treg cells and their fitness, but also in their suppressor function. Furthermore, we found that STAT5 deficiency in Treg cells results in a similar loss of suppressor function and that expression of a constitutively active form of STAT5 can rescue fatal disease resulting from the IL-2R deficiency. These results suggest a key role of IL-2R-STAT5 signaling in linking differentiation and maintenance of Treg cells and their function. STAT5 binds to the Foxp3 promoter and the intronic Foxp3 regulatory element CNS2 and is involved in Foxp3 induction and maintenance³⁸. Runx-CBFβ complexes also bind to CNS2 and the Foxp3 promoter and affect Foxp3 expression levels⁴⁰. While both CNS2- and CBFβ-deficient Treg cells do exhibit reduced Foxp3 expression resembling that of STAT5- or IL-2R-deficient Treg cells, the impairment of suppressor function in the latter was much more severe. Thus, the decrease in Foxp3 expression alone cannot account for a severe loss of Treg cell suppressor function in the absence of STAT5 or IL-2R. Indeed, our analysis of gene expression and functional features imparted upon expression of the active form of STAT5 pointed to a heightened ability of Treg cells to bind to DC and suppress their activation. Furthermore, expression of a constitutively active form of STAT5 partially rescued the near-complete loss of Treg suppressor function in the absence of TCR signaling^{34, 35}. These results may appear at odds with the previous finding that STAT5bCA transgene driven by the proximal lck promoter and Eμ enhancer failed to curtail fatal lymphoproliferative disease in Il2rb−/− mice despite restoring Foxp3 expression and Treg cell differentiation in the thymus⁹. However, the interpretation of the latter result is problematic due to a massive expansion of pre-leukemic T and B cells and reduced expression of the STAT5bCA transgene in peripheral Treg cells.

Our studies clearly demonstrated that IL-2-deprivation by Treg cells was fully dispensable for suppression of IL-2R-sufficient CD4+ T cells even though IL-2R signaling was required. However, IL-2R dependent IL-2 consumption by Treg cells was indispensable for suppression of CD8+ T cell responses. The latter seemingly unexpected finding makes sense in light of the observed exquisite sensitivity of both naïve and activated CD8+ T cells to IL-2 induced stimulation. Furthermore, IL-2 is produced upon activation of both naïve CD4+ and CD8+ T cells within hours after TCR engagement in contrast to effector cytokines such as IL-4 and IFNγ whose production requires Tnaïve cell differentiation into Teff cells on a much longer time scale⁴¹. These distinguishing features provide a likely explanation for a need for a distinct mechanism of control of CD8+ T cell responses by Treg cells through IL-2 consumption.

It has been suggested that sensing of local IL-2 production by Treg cells enables “licensing” of their suppressor function²¹. However, the rescue of suppression of CD4+ T cell responses by IL-2R-deficient Treg cells expressing a constitutively active form of STAT5 suggest that activated Treg cells can suppress autoimmunity without identifying the cellular source of IL-2. Thus, while IL-2 is a booster for Treg cell suppressor function, it may not play an indispensable role as a cue for specific targeting.

Genetically modified T cells are emerging as a potent means of therapy in some forms of cancer. The observed enhanced suppressor activity of Treg cells expressing a constitutively active form of STAT5 and significantly reduced severity of organ-specific autoimmunity in their presence suggest that such a modification of Treg cells may hold promise for an optimal design of Treg cell-based therapies for a variety of autoimmune and inflammatory disorders and in organ transplantation.

Our studies suggest that IL-2R signaling and STAT5 activation potentiates suppression of both CD4+ and CD8+ T cell responses in diverse biological settings and point to a distinct requirement for IL-2R mediated depletion of IL-2 by Treg cells for their control of CD8+ T cell responses. Our findings highlight the central role of IL-2 receptor signaling driven STAT5 activation in supporting and boosting suppressor function of differentiated Treg cells and serving as a nexus linking Treg cell differentiation and maintenance with their suppressor function. In this regard, it is noteworthy that although a Foxp3 ortholog has not been identified in birds, chicken and duck CD4+ T cell subsets expressing high amounts of IL-2Rα chain possess in vitro suppressor activity suggesting the importance of evolutionary conservation of IL-2Rα function in suppressive T cells^{42, 43}.

### Example 3: In Vitro Generation of STAT5-CA Treg

A sample, e.g. blood, containing immune cells is taken from a subject. The immune cells are separated from other components of the sample, e.g, red blood cells and/or serum. The immune cell population is then prepared for separation, e.g. by fluorescence-activated cell sorting, magnetic sorting or other methods known in the art into separate phenotypical components, e.g. naïve, effector memory, central memory, Treg, etc.

A population of Treg cells isolated from the subject is engineered, e.g. by introduction of a heterologous nucleic acid, to express a constitutively active STAT5 protein. Treg cells expressing a constitutively active STAT5 protein are then administered to a subject in need thereof. Alternatively, Treg cells expressing a constitutively active STAT5 protein are expanded in culture prior to administration to a subject in need thereof.

A population of naïve CD4+ T-cells isolated from a subject is cultured under conditions (e.g. plate-bound anti-CD3 and soluble anti-CD28 in the presence of TGF-β) for in vitro generation of Treg. In some embodiments, generated Tregs may be engineered, e.g. by introduction of a heterologous nucleic acid, to express a constitutively active STAT5 protein. In some embodiments, Treg cells expressing a constitutively active STAT5 protein may be administered to a subject in need thereof. Alternatively or additionally, in some embodiments, Treg cells expressing a constitutively active STAT5 protein may be expanded in culture prior to administration to a subject in need thereof.

### Example 4: In Vitro Generation of STAT5-CA CAR-Treg

A Treg cell is engineered, e.g. by introduction of a heterologous nucleic acid, to express a constitutively active STAT5 protein. The Treg cell is further engineered to expresses a chimeric antigen receptor. The CAR-Treg cell is expanded in culture prior to administration to a subject in need thereof. The CAR-Treg cell can be an autologous or heterologous cell with respect to the subject to which the CAR-Treg cell is administered.

### REFERENCES

1. Sakaguchi, S., Sakaguchi, N., Asano, M., Itoh, M. & Toda, M. Immunologic self-tolerance maintained by activated T cells expressing IL-2 receptor alpha-chains (CD25). Breakdown of a single mechanism of self-tolerance causes various autoimmune diseases. J Immunol 155, 1151-1164 (1995).
2. Hori, S., Nomura, T. & Sakaguchi, S. Control of regulatory T cell development by the transcription factor Foxp3. Science 299, 1057-1061 (2003).
3. Fontenot, J. D., Gavin, M. A. & Rudensky, A. Y. Foxp3 programs the development and function of CD4+CD25+ regulatory T cells. Nature immunology 4, 330-336 (2003).
4. Waldmann, T. A. The multi-subunit interleukin-2 receptor. Annual review of biochemistry 58, 875-911 (1989).
5. Furtado, G. C., Curotto de Lafaille, M. A., Kutchukhidze, N. & Lafaille, J. J. Interleukin 2 signaling is required for CD4(+) regulatory T cell function. The Journal of experimental medicine 196, 851-857 (2002).
6. Almeida, A. R., Legrand, N., Papiernik, M. & Freitas, A. A. Homeostasis of peripheral CD4+ T cells: IL-2R alpha and IL-2 shape a population of regulatory cells that controls CD4+ T cell numbers. J Immunol 169, 4850-4860 (2002).
7. Malek, T. R., Yu, A., Vincek, V., Scibelli, P. & Kong, L. CD4 regulatory T cells prevent lethal autoimmunity in IL-2Rbeta-deficient mice. Implications for the nonredundant function of IL-2. Immunity 17, 167-178 (2002).
8. Fontenot, J. D., Rasmussen, J. P., Gavin, M. A. & Rudensky, A. Y. A function for interleukin 2 in Foxp3-expressing regulatory T cells. Nature immunology 6, 1142-1151 (2005).
9. Burchill, M. A., Yang, J., Vogtenhuber, C., Blazar, B. R. & Farrar, M. A. IL-2 receptor beta-dependent STAT5 activation is required for the development of Foxp3+ regulatory T cells. J Immunol 178, 280-290 (2007).
10. Yao, Z. et al. Nonredundant roles for Stat5a/b in directly regulating Foxp3. Blood 109, 4368-4375 (2007).
11. Malek, T. R. & Bayer, A. L. Tolerance, not immunity, crucially depends on IL-2. Nature reviews. Immunology 4, 665-674 (2004).
12. Vang, K. B. et al. IL-2, -7, and -15, but not thymic stromal lymphopoeitin, redundantly govern CD4+ Foxp3+ regulatory T cell development. J Immunol 181, 3285-3290 (2008).
13. Chen, W. et al. Conversion of peripheral CD4+CD25− naïve T cells to CD4+CD25+ regulatory T cells by TGF-beta induction of transcription factor Foxp3. The Journal of experimental medicine 198, 1875-1886 (2003).
14. Malin, S. et al. Role of STAT5 in controlling cell survival and immunoglobulin gene recombination during pro-B cell development. Nature immunology 11, 171-179 (2010).
15. Barron, L. et al. Cutting edge: mechanisms of IL-2-dependent maintenance of functional regulatory T cells. J Immunol 185, 6426-6430 (2010).
16. Setoguchi, R., Hori, S., Takahashi, T. & Sakaguchi, S. Homeostatic maintenance of natural Foxp3(+) CD25(+) CD4(+) regulatory T cells by interleukin (IL)-2 and induction of autoimmune disease by IL-2 neutralization. The Journal of experimental medicine 201, 723-735 (2005).
17. Rubtsov, Y. P. et al. Stability of the regulatory T cell lineage in vivo. Science 329, 1667-1671 (2010).
18. Komatsu, N. et al. Heterogeneity of natural Foxp3+ T cells: a committed regulatory T-cell lineage and an uncommitted minor population retaining plasticity. Proceedings of the National Academy of Sciences of the United States of America 106, 1903-1908 (2009).
19. Hori, S. Lineage stability and phenotypic plasticity of Foxp3(+) regulatory T cells. Immunological reviews 259, 159-172 (2014).
20. Pandiyan, P., Zheng, L., Ishihara, S., Reed, J. & Lenardo, M. J. CD4+CD25+ Foxp3+ regulatory T cells induce cytokine deprivation-mediated apoptosis of effector CD4+ T cells. Nature immunology 8, 1353-1362 (2007).
21. Thornton, A. M., Donovan, E. E., Piccirillo, C. A. & Shevach, E. M. Cutting edge: IL-2 is critically required for the in vitro activation of CD4+CD25+ T cell suppressor function. J Immunol 172, 6519-6523 (2004).
22. Barthlott, T. et al. CD25+CD4+ T cells compete with naïve CD4+ T cells for IL-2 and exploit it for the induction of IL-10 production. International immunology 17, 279-288 (2005).
23. Busse, D. et al. Competing feedback loops shape IL-2 signaling between helper and regulatory T lymphocytes in cellular microenvironments. Proceedings of the National Academy of Sciences of the United States of America 107, 3058-3063 (2010).
24. Yamaguchi, T. et al. Construction of self-recognizing regulatory T cells from conventional T cells by controlling CTLA-4 and IL-2 expression. Proceedings of the National Academy of Sciences of the United States of America 110, E2116-2125 (2013).
25. Smigiel, K. S. et al. CCR7 provides localized access to IL-2 and defines homeostatically distinct regulatory T cell subsets. The Journal of experimental medicine 211, 121-136 (2014).
26. Zambrowicz, B. P. et al. Disruption of overlapping transcripts in the ROSA beta geo26 gene trap strain leads to widespread expression of beta-galactosidase in mouse embryos and hematopoietic cells. Proceedings of the National Academy of Sciences of the United States of America 94, 3789-3794 (1997).
27. Onishi, M. et al. Identification and characterization of a constitutively active STAT5 mutant that promotes cell proliferation. Molecular and cellular biology 18, 3871-3879 (1998).
28. Boyman, O., Kovar, M., Rubinstein, M. P., Surh, C. D. & Sprent, J. Selective stimulation of T cell subsets with antibody-cytokine immune complexes. Science 311, 1924-1927 (2006).
29. Chen, Y. et al. Foxp3(+) regulatory T cells promote T helper 17 cell development in vivo through regulation of interleukin-2. Immunity 34, 409-421 (2011).
30. Cong, Y., Feng, T., Fujihashi, K., Schoeb, T. R. & Elson, C. O. A dominant, coordinated T regulatory cell-IgA response to the intestinal microbiota. Proceedings of the National Academy of Sciences of the United States of America 106, 19256-19261 (2009).
31. Kim, J. M., Rasmussen, J. P. & Rudensky, A. Y. Regulatory T cells prevent catastrophic autoimmunity throughout the lifespan of mice. Nature immunology 8, 191-197 (2007).
32. Su, L. K. et al. Multiple intestinal neoplasia caused by a mutation in the murine homolog of the APC gene. Science 256, 668-670 (1992).
33. Arvey, A. et al. Inflammation-induced repression of chromatin bound by the transcription factor Foxp3 in regulatory T cells. Nature immunology 15, 580-587 (2014).
34. Levine, A. G., Arvey, A., Jin, W. & Rudensky, A. Y. Continuous requirement for the TCR in regulatory T cell function. Nature immunology 15, 1070-1078 (2014).
35. Vahl, J. C. et al. Continuous T cell receptor signals maintain a functional regulatory T cell pool. Immunity 41, 722-736 (2014).
36. Tadokoro, C. E. et al. Regulatory T cells inhibit stable contacts between CD4+ T cells and dendritic cells in vivo. The Journal of experimental medicine 203, 505-511 (2006).
37. Lio, C. W. & Hsieh, C. S. A two-step process for thymic regulatory T cell development. Immunity 28, 100-111 (2008).
38. Feng, Y. et al. Control of the inheritance of regulatory T cell identity by a cis element in the Foxp3 locus. Cell 158, 749-763 (2014).
39. Tran, D. Q. et al. Analysis of adhesion molecules, target cells, and role of IL-2 in human FOXP3+ regulatory T cell suppressor function. J Immunol 182, 2929-2938 (2009).
40. Rudra, D. et al. Runx-CBFbeta complexes control expression of the transcription factor Foxp3 in regulatory T cells. Nature immunology 10, 1170-1177 (2009).
41. Sojka, D. K., Bruniquel, D., Schwartz, R. H. & Singh, N. J. IL-2 secretion by CD4+ T cells in vivo is rapid, transient, and influenced by TCR-specific competition. J Immunol 172, 6136-6143 (2004).
42. Shanmugasundaram, R. & Selvaraj, R. K. Regulatory T cell properties of chicken CD4+CD25+ cells. J Immunol 186, 1997-2002 (2011).
43. Andersen, K. G., Nissen, J. K. & Betz, A. G. Comparative Genomics Reveals Key Gain-of-Function Events in Foxp3 during Regulatory T Cell Evolution. Frontiers in Immunology 3, 113 (2012).
44. Rubtsov, Y. P. et al. Regulatory T cell-derived interleukin-10 limits inflammation at environmental interfaces. Immunity 28, 546-558 (2008).
45. Anders, S. et al. Count-based differential expression analysis of RNA sequencing data using R and Bioconductor. Nature protocols 8, 1765-1786 (2013).
46. Bolger, A. M., Lohse, M. & Usadel, B. Trimmomatic: a flexible trimmer for Illumina sequence data. Bioinformatics 30, 2114-2120 (2014).
47. Love, M. I., Huber, W. & Anders, S. Moderated estimation of fold change and dispersion for RNA-seq data with DESeq2. Genome biology 15, 550 (2014).
48. Tarca, A. L. et al. A novel signaling pathway impact analysis. Bioinformatics 25, 75-82 (2009).
49. Maere, S., Heymans, K. & Kuiper, M. BiNGO: a Cytoscape plugin to assess overrepresentation of gene ontology categories in biological networks. Bioinformatics 21, 3448-3449 (2005).
50. Merico, D., Isserlin, R., Stueker, O., Emili, A. & Bader, G. D. Enrichment map: a network-based method for gene-set enrichment visualization and interpretation. PloS one 5, e13984 (2010).

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. An engineered regulatory T (“Treg”) cell characterized by constitutive STAT activity.

2. The engineered regulatory T cell of claim 1, wherein the regulatory T cell is engineered to constitutively activate a STAT protein.

3. The engineered regulatory T cell of claim 1, wherein the regulatory T cell is engineered to express a higher level or activity of a STAT protein as compared with an appropriate reference.

4. The engineered regulatory T cell of claim 1, wherein the regulatory T cell is engineered to expresses a constitutively active STAT protein.

5. The engineered regulatory T cell of claim 4, wherein the constitutively active STAT protein is or comprises STAT5b.

6. The engineered regulatory T cell of claim 4, wherein the constitutively active STAT protein is constitutively phosphorylated.

7. The engineered regulatory T cell of claim 4, wherein the constitutively active STAT protein is constitutively dimerized.

8. The engineered regulatory T cell of claim 1, wherein the regulatory T cell further expresses a chimeric antigen receptor.

9. The engineered regulatory T cell of claim 1, wherein the regulatory T cell further expresses an endogenous T-cell receptor.

10. A method of treating a subject suffering from an inflammatory or autoimmune disease, disorder, or condition, comprising the step of:
administering to a subject an engineered regulatory T-cell characterized by constitutive STAT activity.

11. The method of claim 10, wherein the method further comprises the steps of:
collecting a sample from a subject containing regulatory T-cells,
isolating regulatory T-cells from the sample,
engineering the regulatory T-cell to comprise constitutive STAT activity,
administering the engineered regulatory T-cell comprising constitutive STAT activity to a subject

12. The method of claim 11, wherein the engineered regulatory T-cell expresses an endogenous T-cell receptor.

13. The method of claim 11, wherein the engineered regulatory T-cell expresses a chimeric antigen receptor.

14. The method of claim 11, wherein the engineered regulatory T-cell is engineered to constitutively activate a STAT protein.

15. The method of claim 11, wherein the engineered regulatory T-cell is engineered to express a higher level or activity of a STAT protein as compared with an appropriate reference.

16. The method of claim 11, wherein the engineered regulatory T-cell is engineered to express a constitutively active STAT protein.

17. The method of claim 16, wherein the constitutively active STAT protein is or comprises STAT5b.

18. The method of claim 16, wherein the constitutively active STAT protein is constitutively phosphorylated.

19. The method of claim 14, wherein the constitutively active STAT protein is constitutively dimerized.

20. The method of claim 11, wherein the subject from whom the sample is collected and the subject to whom the engineered regulatory T-cell is administered are the same.

21. The method of claim 11, wherein the subject from whom the sample is collected and the subject to whom the engineered regulatory T-cell is administered are not the same.

22. The method of claim 10, wherein the method further comprises the steps of:
collecting a sample from a subject containing immune cells,
isolating an immune cell sub-population from the sample,
in vitro generating regulatory T-cells from the isolated immune cell sub-population,
engineering the regulatory T-cell to comprise constitutive STAT activity,
administering the engineered regulatory T-cell comprising constitutive STAT activity to a subject

23. The method of claim 22, wherein the immune cell sub-population consists of naïve CD4+ cells.

24. The method of claim 22, wherein the engineered regulatory T-cell expresses an endogenous T-cell receptor.

25. The method of claim 22, wherein the engineered regulatory T-cell expresses a chimeric antigen receptor.

26. The method of claim 22, wherein the engineered regulatory T-cell is engineered to constitutively activate a STAT protein.

27. The method of claim 22, wherein the engineered regulatory T-cell is engineered to express a higher level or activity of a STAT protein as compared with an appropriate reference.

28. The method of claim 22, wherein the engineered regulatory T-cell is engineered to express a constitutively active STAT protein.

29. The method of claim 28, wherein the constitutively active STAT protein is or comprises STAT5b.

30. The method of claim 28, wherein the constitutively active STAT protein is constitutively phosphorylated.

31. The method of claim 28, wherein the constitutively active STAT protein is constitutively dimerized.

32. The method of claim 22, wherein the subject from whom the sample is collected and the subject to whom the engineered regulatory T-cell is administered are the same.

33. The method of claim 22, wherein the subject from whom the sample is collected and the subject to whom the engineered regulatory T-cell is administered are not the same.
